(19) United States
(12) Patent Application Publication
(10) Pub. No.: US 2025/0090690 A1
(43) Pub. Date: Mar. 20, 2025

(54) METHODS AND SYSTEMS FOR CORRECTING MUTATIONS IN PRPH2

(71) Applicant: The Trustees of Columbia University in the City of New York, New York, NY (US)

(72) Inventors: Yi-Ting Tsai, New York, NY (US); Salvatore M. Caruso, New York, NY (US); Bruna Lopes da Costa, New York, NY (US); Stephen H. Tsang, New York, NY (US); Peter M.J. Quinn, New York, NY (US)

(21) Appl. No.: 18/946,509

(22) Filed: Nov. 13, 2024

Related U.S. Application Data
(63) Continuation of application No. PCT/US2023/066953, filed on May 12, 2023.
(60) Provisional application No. 63/498,373, filed on Apr. 26, 2023.
(60) Provisional application No. 63/341,739, filed on May 13, 2022.

(51) Int. Cl. A61K 48/00; A61K 38/17; A61P 27/02; C12N 9/12; C12N 9/22; C12N 15/11

(52) U.S. Cl. CPC A61K 48/0058; A61K 38/1709; A61P 27/02; C12N 9/1276; C12N 9/22; C12N 15/111; C12N 2310/20; C12Y 207/07049

(57) ABSTRACT

The present disclosure provides systems, methods, and compositions for prime-editing modification of c.828 splice site mutations in the peripherin-2 gene. Particularly the present disclosure provides systems, methods, and compositions for correcting one or more disease-causing splice site mutations selected from: c.828+3A>T, c.828+1G>A, c.828+2T>C, c.828+1G>T, and combinations thereof.

### CROSS-REFERENCE TO RELATED APPLICATIONS

This application is a continuation of International Application No. PCT/US2023/066953, filed May 12, 2023, which claims the benefit of U.S. Provisional Application Nos. 63/341,739, filed May 13, 2022, and 63/498,373, filed Apr. 26, 2023, the contents of which are herein incorporated by reference in their entirety.

### SEQUENCE LISTING

The contents of the electronic sequence listing titled “40853_601_SequenceListing.xml” (Size: 156,177 bytes; and Date of Creation: May 11, 2023) is herein incorporated by reference in its entirety.

### FIELD

The present invention relates to systems, methods, and compositions for prime-editing modification of c.828 splice site mutations in the peripherin-2 gene.

### BACKGROUND

The human peripherin-2 gene (PRPH2), also known as retinal degeneration slow (RDS), is located on chromosome 6 and encodes a transmembrane protein localized to the outer segment of rod and cone photoreceptor cells. PRPH2 aids in the formation, maintenance, and renewal of the outer segment discs. As such, mutations in PRPH2 lead to the disorganization or absence of photoreceptor outer segments. PRPH2 mutations have a frequency in the United States of 1/54,062 and accounts for 5-10% of autosomal dominant retinitis pigmentosa (adRP). As of March 2022, there are over three hundred unique PRPH2 variants. The second most reported variant being c.828+3A>T. Two rarer c.828 splice site mutations have also been reported, c.828+1G>A and c.828+2T>C. Traditional base editing only allows for C to T and A to G base transitions and more recently C to G transversions.

PRPH2 patients present with a wide range of phenotypes. The gene has been linked to clinical manifestations including adRP, adult vitelliform macular dystrophy, pattern macular dystrophy, fundus flavimaculatus-like dystrophy, central areolar choroidal dystrophy, and cone-rod dystrophy. The complex nature of peripherin-2 protein function and regulation, combined with variability in disease mechanism, has impeded development of clinically viable PRPH2 genetic treatments. Accordingly, there is no successful treatment for PRPH2-mediated inherited retinal diseases.

### SUMMARY

Provided herein are methods for correcting one or more splice site mutations in the peripherin-2 gene. In some embodiments, the methods comprise contacting a DNA encoding the peripherin-2 gene with: a Cas protein; a reverse transcriptase; one or more RNA polynucleotides comprising a spacer sequence and an extension sequence comprising a primer binding sequence (PBS) and a reverse transcriptase template (RTT) sequence; and, optionally, a nicking guide RNA (ngRNA). In some embodiments, the RTT sequence encodes one or more nucleotides to correct the one or more splice site mutations in the peripherin-2 gene.

In some embodiments, the one or more splice site mutations are selected from: c.828+3A>T, c.828+1G>A, c.828+2T>C, and combinations thereof. In some embodiments, the RTT sequence is configured to correct two or more of the one or more splice site mutations. In some embodiments, the RTT sequence is configured to correct two or more of or all of: c.828+3A>T, c.828+1G>A, and c.828+2T>C. In some embodiments, the RTT sequence corrects the c.828+1G>T splice site mutation.

In some embodiments, Cas protein is Cas9 or a variant or fragment thereof. In some embodiments, the Cas protein is a Cas9 nickase. In some embodiments, the Cas protein comprises a Cas protein variant configured to target an expanded range of protospacer adjacent motif (PAM) sequences. In some embodiments, the Cas protein comprises a variant of the Streptococcus pyogenes Cas9 selected from xCas9, Cas9-VQR, SpG and SpRY. In some embodiments, the Cas protein and the reverse transcriptase are in a single fusion protein.

In some embodiments, the spacer sequence and the extension sequence are contained within a single RNA polynucleotide.

In some embodiments, the spacer sequence, the extension sequence, the ngRNA, or any combination thereof comprises any of the associated sequences as disclosed herein.

In some embodiments, the DNA encoding the peripherin-2 gene is in a cell. In some embodiments, the cell is a eukaryotic cell. In some embodiments, the cell is a human cell. In some embodiments, the cell is in vitro. In some embodiments, the cell is ex vivo. In some embodiments, the cell is in vivo. In some embodiments, the DNA encoding the peripherin-2 gene is genomic DNA.

In some embodiments, the contacting comprises introducing into the cell: a Cas protein, or a nucleic acid encoding thereof; a reverse transcriptase, or a nucleic acid encoding thereof; a spacer sequence, primer binding sequence (PBS), and a reverse transcriptase template (RTT) sequence, or one or more nucleic acids encoding thereof; and, optionally, a nicking guide RNA (ngRNA), or a nucleic acid encoding thereof. In some embodiments, introducing into the cell comprises administering to the subject.

Also provided herein are systems for modifying the peripherin-2 gene. In some embodiments, the systems are for use in treating or preventing a disease or disorder caused or mitigated by mutations in peripherin-2 gene. In some embodiments, the systems are for use in correcting one or more splice site mutations in the peripherin-2 gene.

In some embodiments, the systems comprise: a Cas protein, or a nucleic acid encoding thereof; a reverse transcriptase, or a nucleic acid encoding thereof; one or more RNA polynucleotides comprising a spacer sequence and an extension sequence comprising a primer binding sequence (PBS) and a reverse transcriptase template (RTT) sequence; or one or more nucleic acids encoding thereof; and, optionally, a nicking guide RNA (ngRNA), or a nucleic acid encoding thereof.

In some embodiments, the RTT sequence encodes one or more base substitutions to be introduced into the peripherin-2 gene. In some embodiments, the spacer sequence and the extension sequence are contained within a single RNA polynucleotide.

In some embodiments, Cas protein is Cas9 or a variant or fragment thereof. In some embodiments, the Cas protein is a Cas9 nickase. In some embodiments, the Cas protein comprises a Cas protein variant configured to target an expanded range of PAM sequences. In some embodiments, the Cas protein comprises a variant of the Streptococcus pyogenes Cas9 selected from xCas9, Cas9-VQR. SpG and SpRY. In some embodiments, the Cas protein and the reverse transcriptase are in a single fusion protein. In some embodiments, the Cas protein and the reverse transcriptase are provided as a split intein system.

In some embodiments, the spacer sequence, the extension sequence, the ngRNA, or any combination thereof comprises any of the associated sequences as disclosed herein.

In some embodiments, the peripherin-2 gene is a mutant peripherin-2 gene comprising one or more disease-causing mutations. In some embodiments, the one or more base substitutions correct one or more splice site mutations in the human peripherin-2 gene. In some embodiments, the one or more base substitutions correct any or all of the following mutations: c.828+3A>T, c.828+1G>A, c.828+1G>T, and c.828+2T>C. In some embodiments, the one or more base substitutions corrects the c.828+1G>T mutation.

In some embodiments, the peripherin-2 gene is a wild-type peripherin-2 gene. In some embodiments, the one or more base substitutions install one or more splice site mutations in the peripherin-2 gene. In some embodiments, the one or more splice site mutations comprise one or more disease-causing mutations.

Provided herein are methods for modifying a peripherin-2 gene comprising contacting a DNA encoding the peripherin-2 gene with a system disclosed herein, or the components thereof, to the subject.

In some embodiments, the peripherin-2 gene comprises one or more splice site mutations. In some embodiments, the RTT sequence encodes one or more nucleotides to correct the one or more splice site mutations in the peripherin-2 gene. In some embodiments, the one or more splice site mutations include any or all of the following splice site mutations: c.828+3A>T, c.828+1G>A, c.828+1G>T, and c.828+2T>C. In some embodiments, the RTT sequence is configured to correct all of the one or more splice site mutations in the peripherin-2 gene.

In some embodiments, the DNA encoding the peripherin-2 gene is in a cell. In some embodiments, the cell is a eukaryotic cell. In some embodiments, the cell is a human cell. In some embodiments, the cell is in vitro. In some embodiments, the cell is ex vivo. In some embodiments, the cell is in vivo. In some embodiments, the DNA encoding the peripherin-2 gene is genomic DNA.

In some embodiments, the contacting comprises introducing into the cell: a Cas protein, or a nucleic acid encoding thereof; a reverse transcriptase, or a nucleic acid encoding thereof; a spacer sequence, primer binding sequence (PBS), and a reverse transcriptase template (RTT) sequence, or one or more nucleic acids encoding thereof; and, optionally, a nicking guide RNA (ngRNA), or a nucleic acid encoding thereof. In some embodiments, introducing into the cell comprises administering to the subject.

Further provided herein are methods of treating or preventing a disease or disorder in a subject. In some embodiments, the methods comprise administering a system disclosed herein, or the components thereof, to the subject.

In some embodiments, the disease or disorder is caused or mitigated by mutations in peripherin-2 gene. In some embodiments, the disease or disorder comprises retinal degeneration, retinitis pigmentosa, macular degeneration, macular dystrophy, fundus flavimaculatus-like dystrophy, central areolar choroidal dystrophy, cone-rod dystrophy, or a combination thereof.

In some embodiments, the system is configured for delivery to retinal cells. In some embodiments, the system is configured for delivery to rod and cone photoreceptor cells.

In some embodiments, the subject has one or more splice site mutations in the peripherin-2 gene. In some embodiments, the subject has a peripherin-2 gene with any or all of the following mutations c.828+3A>T, c.828+1G>A, and c.828+2T>C. In some embodiments, the subject has a peripherin-2 gene with a c.828+1G>T mutation.

Additionally provided are methods for correcting two or more mutations in a gene comprising contacting the gene with a prime editing composition comprising one or more pegRNAs configured to correct any or all of the two or more mutations. In some embodiments, the prime editing composition comprises a single pegRNA. In some embodiments, the composition further comprises any or all of: a nicking guide RNA (ngRNA), a Cas9 nickase, and a reverse transcriptase.

Other aspects and embodiments of the disclosure will be apparent in light of the following detailed description and related figures.

### BRIEF DESCRIPTION OF THE FIGURES

FIGS. 1A-1D are color fundus and autofluorescence imaging of a patient with a single pathogenic variant in PRPH2. FIGS. 1A and 1B are color fundus photographs of the right and left eye, respectively, demonstrate widespread RPE atrophy with a central area of macular atrophy sparing the fovea. There is sparse intraretinal pigment migration OS>OD in the inferotemporal periphery. FIGS. 1C and 1D are fundus autofluorescence imaging of the right and left eye, respectively, reveal scattered hypoautofluorescence atrophic lesions OS>OD extending throughout the periphery with relative sparing of the superior retina.

FIGS. 2A and 2B show the prime editing strategy and next generation sequencing results for PRPH2 c.828+1 G>A installation. FIG. 2A is a schematic detailing the native, healthy wild-type genomic sequence (SEQ ID NO: 126, showing the respective amino acid sequence of SEQ ID NO: 127)) and the prime editing strategy responsible for generating the PRPH2 c.828+1 G>A disease model in HEK293 cells (pegRNA spacer-SEQ ID NO: 128; nsgRNA1-SEQ ID NO: 129; and installation 3′ extension-SEQ ID NO: 130). Directional protospacer sequences, respective cut sites, and intended mutation are annotated. FIG. 2B is next generation sequencing results generated from triple plasmid transfection of HEK293 cells with PegRNA, PE machinery, and ngRNA plasmids. Reference sequence is the wild-type sequence (SEQ ID NO: 131), arrow points to the precise edited sequence, bold lettering indicates substitution, boxes indicate random insertions. Results indicate successful installation (SEQ ID NOs: 131-137, top to bottom), which was then selected to establish isolated disease model.

FIGS. 3A-3B show the evaluation of PE2 VS PE3 systems for PRPH2 c.828+1 G>A installation. To evaluate the long-term editing efficiency for the PRPH2 c.828+1 G>A installation, two transfections were performed in parallel on health HEK293 cells: a PE2 strategy using only the PE machinery and pegRNA, and a PE3 strategy using identical plasmids to PE2 and an additional ngRNA plasmid. Time course results are shown below on days 3, 7, and 10 (FIG. 3A). Results and calculations are summarized in the table in FIG. 3B.

FIG. 4A is a schematic of exemplary prime editing strategies for correction of the PRPH2 c.828+1, +2 and +3 mutations individually (“only”) or a strategy to use for every mutation (“PAN”). PRPH2 DNA segment-SEQ ID NO: 138; amino acid segment-SEQ ID NO: 139. FIG. 4B is a schematic of exemplary designs for PAN correction. PRPH2 DNA segments-SEQ ID NO: 140, top, and SEQ ID NO: 142, bottom; amino acid segments-SEQ ID NO: 141, top, and SEQ ID NO: 143, bottom.

FIGS. 5A and 5B are charts of the efficiency of c.828+1G>A NGG and NGA PAM prime editing correction, respectively, with various a prime editing guide RNA (pegRNA) and nicking guide RNA (ngRNA) combinations.

FIGS. 6A and 6B are charts of the efficiencies of correction of c.828+3A>T with combinations of pegRNA and ngRNA.

FIGS. 7A-7D show the efficiency of correction of the +3 mutation (FIGS. 7A and 7C) and the +1 mutation (FIGS. 7B and 7D) for the NGA and NGG PAM prime editing strategies. respectively.

FIG. 8 is Netgene2 analysis of donor splice sites for PRPH2. Blue (17971) marks canonical donor splice site. Red (18000) marks cryptic splice site. Asterisk highlights reduced confidence of canonical splice site.

FIG. 9 is a schematic of the prime editing mechanism. (1) The spacer anneals with its complementary strand of DNA. (2) nCas9 creates a nick on the PAM-containing strand of the target DNA. (3) The PBS anneals with the 3′overhang of the PAM-containing strand. (4) RTase starts extending the 3′ end based on the RT template.

FIGS. 10A-10C show PRPH2 c.828 splice site HEK293 knock-in lines. FIG. 10A shows PRPH2 c.828+1G>A (SEQ ID NO: 150) and c.828+3A>T (SEQ ID NO: 152) single cell isolated and expanded clones. PRPH2 c.828+2T>C (SEQ ID NO: 151) is from bulk cell sequencing. Wild-type PRPH2 DNA sequence segment (SEQ ID NO: 148) and corresponding protein sequence (SEQ ID NO: 149) shown for reference. FIG. 10B shows aberrantly spliced transcript in PRPH2 c.828 splice site knock-in HEK293 lines. Primers amplifying exon 2/3 boundary of PRPH2 from cDNA template show aberrant splicing in the c.828 splice site knock-in HEK293 lines compared to wild type. Wild-type—SEQ ID No: 155; c.828+1G>A-SEQ ID NO: 156; and c.828+3A>T-SEQ ID NO: 157. Aligned sanger traces of PCR products confirm inclusion of 29 bp of intron 2 in knock-in lines. *Snapgene misaligned wild type by 1 bp. Unspliced PRPH2 DNA sequence segment (SEQ ID NO: 153) and corresponding protein sequences (SEQ ID NO: 154 and 158) shown for reference. FIG. 10C shows that PRPH2 c.828 splice site mutations are predicted to generate truncated protein. PRPH2 c. 828+1G>A DNA is SEQ ID NO: 159 and amino acid is SEQ ID NO: 160; c. 828+1G>T DNA is SEQ ID NO: 161 and amino acid is SEQ ID NO: 162; c. 828+2T>C DNA is SEQ ID NO: 163 and amino acid is SEQ ID NO: 164; c. 828+3A>T DNA is SEQ ID NO: 165 and amino acid is SEQ ID NO: 166.

FIGS. 11A and 11B show an alternative representation of the data from mutation specific approaches as in FIGS. 5A and 5B, respectively. FIG. 11A shows correction of PRPH2 c.828+1G>A knock-in line using an NGA based spacer for the pegRNA. FIG. 11B shows correction of PRPH2 c.828+1G>A knock-in line using an NGG based spacer for the pegRNA.

FIGS. 12A and 12B show the PAN mutation approach for correction of PRPH2 c.828+1G>A and c.828+3A>T knock-in lines using an NGG based spacer (FIG. 12A) or an NGA based spacer (FIG. 12B) for the pegRNA. FIGS. 12A and 12B are alternative representation of the data from FIGS. 7A-7D.

FIG. 13 shows NGG c.828 PAN Split Intein Prime Editing corrects the c.828+1G>A PRPH2 mutation (SEQ ID NO: 167 for reference and untreated). Transfection of the c.828+1G>A HEK293 knock-in line with the NGG c.828 PAN split intein plasmids leads to correction of the mutation (SEQ ID NO: 169 for treated). Sanger of bulk HEK293 cells is shown. Amino acid sequence segment is SEQ ID NO: 168.

FIG. 14 shows NGG c.828 PAN Split Intein Prime Editing restores canonical mRNA splicing in HEK293 c.828+1G>A PRPH2 knock-in line. Primers amplify exon 2/3 boundary of PRPH2 from cDNA template from PE treated and untreated c.828+1G>A knock-in HEK293. A1 and B1 bands represent aberrant splicing due to the c.828+1G>A mutation (SEQ ID NO: 173 and 174, respectively). B2 represents restored canonical splicing due to therapeutic prime editing (SEQ ID No: 175). Reference DNA sequence is SEQ ID NO: 170 with two amino acid sequence segments of SEQ ID NO: 171 and 172.

FIGS. 15A-15B show AAV transduction of retinal organoid cell types with a photoreceptor specific promoter (GRK1) at Day 290. Recoverin marks maturating photoreceptors. ONL: outer nuclear layer.

FIGS. 16A and 16B show PRPH2 localizes to the photoreceptor outer segments. FIG. 16A is an image of an adult cadaveric human retina. FIG. 16B is an image of a day 180 control retinal organoid. ONL: outer nuclear layer, INL: inner nuclear layer, GCL: ganglion cell layer.

FIGS. 17A and 17B show PRPH2 c.828+1G>A patient iPSC-derived retinal organoids. FIG. 17A is a brightfield image of day 50 patient retinal organoid. FIG. 17B is a Day 100 patient retinal organoid. Recoverin marks maturating photoreceptors. β-catenin marks the adherens junctions.

FIG. 18 is a PRPH2 c.828+1G>A patient iPSC-derived retinal organoids have aberrant PRPH2 mRNA splicing. Primers amplifying exon 2/3 boundary of PRPH2 from cDNA template from day 100 PRPH2 c.828+1G>A patient iPSC-derived retinal organoids. The heterozygous patient retinal organoid had both canonical and aberrantly spliced PRPH2 mRNA.

### DETAILED DESCRIPTION

To date there is no treatment available for PRPH2-mediated inherited retinal diseases. Several therapeutic modalities have been explored, including AAV and nanoparticle mediated gene augmentation, gene knockdown and replacement, and the use of neurotrophic factors. The cDNA of PRPH2 is small, approximately 1.1 kb, making it ideal for AAV-mediated gene augmentation. Gene augmentation for PRPH2-associated inherited retinal diseases has led to both retinal structural and functional improvements in mouse models. However, studies have shown that PRPH2 overexpression effects are transient or required precise dosing of PRPH2 due to the severe haploinsufficiency phenotype. Many PRPH2 mutations are gain-of-function or act in a dominant negative manner and therefore other approaches are required to eliminate the mutant allele. As proof-of-principal, gene knockdown of Prph2 using small interfering RNA (siRNA) and then replacement with a wildtype but siRNA resistant copy of Prph2 has been evaluated in wild type mouse retina using AAV-mediated delivery. While siRNA delivery alone led to a decrease in electroretinography (ERG) response, co-delivery prevented this functional decrease in retinal response but only partial recovery of total Prph2 levels. A similar study was done via electroporation of constructs in mouse wild type retinal explants showed recovery of Prph2 mRNA levels when using siRNA resistant Prph2 co-delivered with a siRNA shown to efficiently knockdown Prph2 mRNA levels. These methodologies showed promise but have not been further explored in a Prph2 disease models. Additionally, several teams have tried non-gene-specific therapies to treat Prph2-IRD mouse models using neurotrophic factors such as CTNF, FGF2, PEDF or nontraditional neurotrophic factors such as erythropoietin and nilvadipine. In several cases some structural or functional improvements were detected in the treated Prph2-IRD mouse models, however expression of CTNF also led to altered retinal organization and gene expression. Efficient base editing is unfeasible for the prevalent PRPH2 c.828+3A>T and the rare c.828+1G>T mutations. In addition, the c.828+1G>A and c.828+2T>C PRPH2 mutations may be affected by the presence of bystanders and/or the need to use a near-PAMless Cas9, that can lead to lower editing efficiency and higher off-targeting.

The disclosed systems, compositions, and methods provide methods to correct the +1, +2 and +3 c.828 PRPH2 patient mutations separately or together in any combination. Existing base editors can only create a subset of changes (C->T, G->A, A->G, and T->C) and are less precise, resulting in the undesired introduction of mutations within an editing window of the target nucleic acid. The disclosed systems, compositions, and methods support prime editing based correction of the +1, +2 and/or +3 c.828 splice site mutations utilizing engineered combinations of pegRNAs and nicking sgRNAs. The systems, compositions, and methods can correct one or more of the mutations without requiring prior knowledge of which mutation or mutations are present.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the terms “administering,” “providing,” and “introducing,” are used interchangeably herein and refer to the placement into a subject by a method or route which results in at least partial localization to a desired site. Administration can be by any appropriate route which results in delivery to a desired location in the subject.

The term “contacting” as used herein refers to bring or put in contact, to be in or come into contact. The term “contact” as used herein refers to a state or condition of touching or of immediate or local proximity.

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corcy, Biochemistry, 41 (14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

As used herein, the term “preventing” refers to partially or completely delaying onset of a disease, disorder and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition.

As used herein, “treat,” “treating,” and the like means a slowing, stopping, or reversing of progression of a disease or disorder. The term also means a reversing of the progression of such a disease or disorder. As such, “treating” means an application or administration of the methods or devices described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of devices and systems contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods herein, the mammal is a human.

A “vector” or “expression vector” is a replicon, such as plasmid, phage, virus, or cosmid, to which another DNA segment, e.g., an “insert,” may be attached or incorporated so as to bring about the replication of the attached segment in a cell.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (e.g., altered characteristics) when compared to the wild-type gene or gene product. It is noted that naturally-occurring mutants can be isolated; these are identified by the fact that they have altered characteristics when compared to the wild-type gene or gene product.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Prime Editing

Prime editing is a double-strand break (DSB)-independent clustered-regularly interspaced short palindromic repeats (CRISPR)/CRISPR-associated (Cas) system that can ameliorate both transition and transversion mutations in addition to small deletions and insertions. Generally, a prime editing guide RNA (pegRNA) is used in conjunction with a prime editor, e.g., a H840A Streptococcus pyogenes Cas9 (spCas9) nickase linked to an optimized Moloney murine leukemia virus (MMLV) reverse transcriptase (RT).

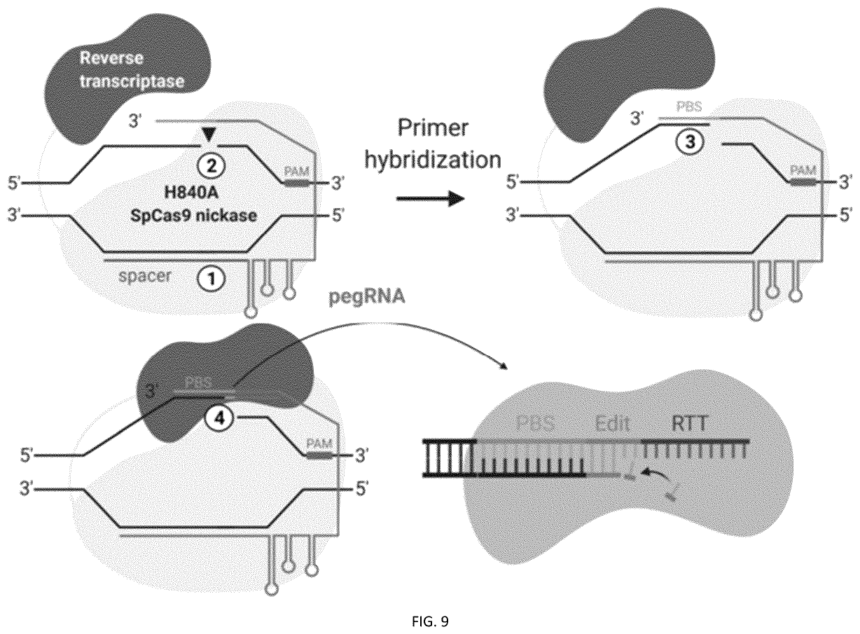

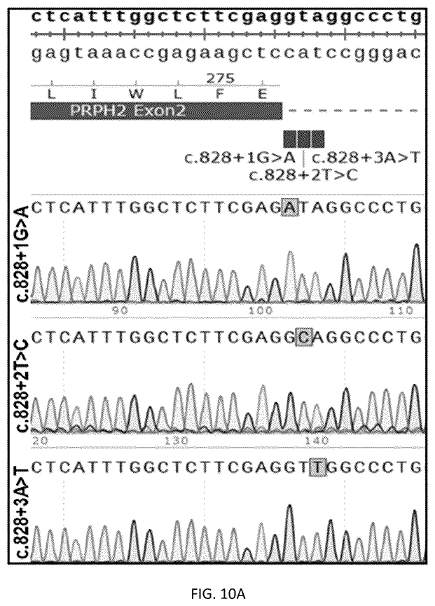

pegRNAs are similar to standard single-guide RNAs (sgRNAs) but differ due to a 3′ extension sequence comprising a primer binding site (PBS) and an adjacent reverse transcription template (RTT) sequence. The primer binding site hybridizes with the bases upstream of the prime editor generated nick, while the RTT encodes the information of the intended edits and directs reverse transcription. Together, the prime editor and the pegRNA form the prime editing 2 strategy (PE2). The Cas9 nickase is guided to the DNA target site by the pegRNA. After nicking by Cas9, the reverse transcriptase uses the pegRNA to template reverse transcription of the desired edit, directly polymerizing DNA onto the nicked target DNA strand. The edited DNA strand replaces the original DNA strand, creating a heteroduplex containing one edited strand and one unedited strand. Lastly, the prime editor guides resolution of the heteroduplex to favor copying the edit onto the unedited strand, completing the process. Once the prime editor incorporates the edit into one strand, there is a mismatch between the original sequence on one strand and the edited sequence on the other strand. In some embodiments, an additional nicking guide RNA (ngRNA) is used to nick the non-edited strand, directing DNA repair enzymes to use the edited strand as a template to remake the mismatched strand. The prime editor, the pegRNA, and ngRNA form prime editing 3 (PE3) strategies. A schematic of an exemplary prime editing mechanism is shown in FIG. 9.

Disclosed herein are methods and systems for modifying (e.g., correcting one or more splice site mutations) the human peripherin-2 gene by prime editing. The methods and systems comprise a sequence-specific nuclease, or a nucleic acid encoding thereof; an RNA-dependent DNA polymerase, or a nucleic acid encoding thereof; one or more RNA polynucleotides comprising a spacer sequence and an extension sequence comprising a primer binding sequence (PBS) and a reverse transcriptase template (RTT) sequence, or one or more nucleic acids encoding thereof; and optionally, a nicking guide RNA (ngRNA), or a nucleic acid encoding thereof.

In some embodiments, the RTT sequence encodes one or more nucleotides to modify the peripherin-2 gene. In some embodiments, the RTT sequence is configured to correct all of the one or more splice site mutations. In some embodiments, the RTT sequence is configured to correct all of the one or more of: c.828+3A>T, c.828+1G>A, c.828+1G>T, and c.828+2T>C. In some embodiments, the RTT encodes a wild-type sequence of the peripherin-2 gene.

In some embodiments, the RTT sequence is configured to install one or more splice site mutations in the peripherin-2 gene. For example, when the peripherin-2 gene is a wild-type peripherin-2 gene and the RTT sequence may be configured to install one or more splice site mutations in the wild-type peripherin-2 gene. As such, the disclosed methods can be used to generate disease models for splice site mutations in the peripherin-2 gene.

In some embodiments, the methods comprise contacting the DNA encoding the peripherin-2 gene with the disclosed system. In some embodiments, the methods comprise contacting the DNA encoding the peripherin-2 gene with a Cas9 protein; a reverse transcriptase; one or more RNA polynucleotides comprising a spacer sequence and an extension sequence comprising a primer binding sequence (PBS) and a reverse transcriptase template (RTT) sequence; and optionally, a nicking guide RNA (ngRNA). In some embodiments, the RTT sequence encodes one or more nucleotides to correct the one or more splice site mutations in the peripherin-2 gene. In some embodiments, the RTT encodes a wild-type sequence of the peripherin-2 gene. In some embodiments, the RTT sequence encodes one or more nucleotides to generate or install one or more splice site mutations in the peripherin-2 gene. In some embodiments, the RTT encodes a mutant sequence of the peripherin-2 gene.

In some embodiments, the DNA encoding the peripherin-2 gene is in a cell. In some embodiments, the cell is a eukaryotic cell. In some embodiments, the cell is a human cell. In some embodiments, the cell is in vitro. In some embodiments, the cell is ex vivo. In some embodiments the cell is in vivo. In some embodiments, the DNA encoding the peripherin-2 gene is genomic DNA. In some embodiments, the peripherin-2 gene is the human peripherin-2 gene.

In some embodiments, contacting the DNA encoding the peripherin-2 gene comprises introducing into the cell: a nucleic acid encoding a Cas9 protein, a nucleic acid encoding a reverse transcriptase; and a nucleic acid encoding one or more RNA polynucleotides comprising a spacer sequence and an extension sequence comprising a primer binding sequence (PBS) and a reverse transcriptase template (RTT) sequence; and, optionally, a nucleic acid encoding a nicking guide RNA (ngRNA). In some embodiments, a single nucleic acid encodes the Cas9 protein and the reverse transcriptase. In some embodiments, a single nucleic acid encodes the spacer sequence, the extension sequence and, optionally, the ngRNA.

In some embodiments, introducing into the cell comprises administering to the subject.

### 1. Sequence-Specific Nuclease

Exemplary sequence-specific nucleases for use in the present invention include, but are not limited to, Cas proteins, Argonaute (Ago) proteins, zinc finger nucleases (ZFNs), and transcription activator-like effector nucleases (TALEN). In some embodiments, the sequence-specific nuclease is a Cas protein.

Cas proteins are described in further detail in, e.g., Haft et al., PLOS Comput. Biol., 1(6): e60 (2005), incorporated herein by reference. The Cas protein may be any Cas endonuclease, or fragment or naturally-occurring or engineered variants thereof. In some embodiments, the Cas endonuclease is a Class 2 Cas endonuclease. In some embodiments, the Cas endonuclease is a Type V Cas endonuclease. In some embodiments, the Cas protein is Cas9, Cas12a, otherwise referred to as Cpf1, or Cas14. In one embodiment, the Cas9 protein is a wild-type Cas9 protein. In some embodiments, the Cas9 protein is a Cas9 variant.

The Cas9 protein can be obtained or derived from any suitable microorganism, and a number of bacteria express Cas9 protein orthologs or variants. In some embodiments, the Cas9 is from Streptococcus pyogenes or Staphylococcus aureus. Cas9 proteins of other species are known in the art (see, e.g., U.S. Patent Application Publication 2017/0051312, incorporated herein by reference) and may be used in connection with the present disclosure. The amino acid sequences of Cas proteins from a variety of species are publicly available through the GenBank and UniProt databases.

In certain embodiments, a Cas nuclease can only cleave a target sequence if an appropriate PAM is present. See, for example Doudna et al., Science, 2014, 346 (6213): 1258096, incorporated herein by reference. A PAM site is a nucleotide sequence in proximity to a target sequence. For example, PAM site may be a DNA sequence immediately following the DNA sequence targeted by the Cas protein. A PAM can be 5′ or 3′ of a target sequence. A PAM can be upstream or downstream of a target sequence. A PAM can be 1, 2, 3, 4, 5, 6, 7, 8, 9, 10 or more nucleotides in length. In certain embodiments, a PAM is between 2-6 nucleotides in length. Non-limiting examples of the PAM sequences include: CC, CA, AG, GT, TA, AC, CA, GC, CG, GG, CT, TG, GA, AGG, TGG, T-rich PAMs (such as TTT, TTG, TTC, etc.), NGG, NGA, NAG, and NGGNG, where “N” is any nucleotide.

In some embodiments, the Cas protein comprises a Cas variant configured to target an expanded or altered range of PAM sequences which may facilitate essentially PAMless cleavage. In some embodiments, the Cas protein comprises a variant of the Streptococcus pyogenes Cas9 enzyme selected from xCas9. Cas9-VQR. SpG and SpRY. See, for example, Walton et al., Science. 2020 Apr. 17; 368 (6488): 290-296, Hu, et al., Nature 2018; 556 (57-63), Kleinstiver et al., Nature 2015; 523 (7561): 481-5, Hu et al., Mol Plant 2016; 9, 43-945, incorporated herein by reference in their entirety.

In some embodiments, the Cas protein is a Cas9 nickase (Cas9n). Wild-type Cas9 has two catalytic nuclease domains facilitating double-stranded DNA breaks. A Cas9 nickase protein is typically engineered through inactivating point mutation(s) in one of the catalytic nuclease domains causing Cas9 to nick or enzymatically break only one of the two DNA strands using the remaining active nuclease domain. Cas9 nickases are known in the art (see, e.g., U.S. Patent Application Publication 2017/0051312, incorporated herein by reference) and include, for example, Streptococcus pyogenes with point mutations at D10 or H840.

### 2. RNA-Dependent DNA Polymerase

RNA-dependent DNA polymerases (e.g., reverse transcriptases) synthesize complementary DNA using RNA as a template. Any RNA-dependent DNA polymerase, or variant or truncation thereof or enzyme having RNA-dependent DNA polymerase activity can be utilized in the systems and methods herein. Exemplary RNA-dependent DNA polymerases include retroviral reverse transcriptases, retrotransposon reverse transcriptases, bacterial reverse transcriptases, Tth DNA polymerase, Taq DNA polymerase, Tma DNA polymerase, and functional variants or fragments thereof. In some embodiments, the RNA-dependent DNA polymerases is a Moloney murine leukemia virus (MMLV) reverse transcriptase.

In some embodiments, the reverse transcriptase and the sequence-specific nucleases (e.g., Cas protein) comprise a fusion protein, also referred to herein as a prime editor. The reverse transcriptase can be fused to the sequence-specific nucleases (e.g., Cas protein) in any orientation and may be separated from the sequence-specific nucleases (e.g., Cas protein) with an amino acid linker.

In some embodiments, the reverse transcriptase and the sequence-specific nuclease (e.g., Cas protein) are provided in a split system. For example, the reverse transcriptase and the sequence-specific nuclease are provided as two or more different polypeptides (or nucleic acids encoding the two or more different polypeptides) such that two or more separate polypeptides together form a functional fusion protein or prime editor. In some embodiments, the sequences that encode the two or more separate polypeptides are present on the same vector. In some embodiments, they are present on two or more separate vectors. A split system can be used for any number of reasons such as overcoming a packing limit of vector or other delivery vehicle or regulating the active system by temporal or spatial introduction of the two or more vectors. Split systems include, but are not limited to, intein, MS2 or SunTag based systems. The split system may comprise more than one split system type (e.g., an intein based system and a SunTag based system) or more than one split system of a single type (e.g., one or more intein based systems).

In some embodiments, the split system is a split intein system. A “split intein” involves two or more complementary part inteins, for example two or more pairs of an N-intein and C-intcin, that associate selectively and extremely tightly to form a full intein. As used herein, the word “intein” means a naturally-occurring or artificially-constructed polypeptide sequence embedded within a precursor protein that can catalyze a splicing reaction during post-translation processing of the protein. A list of known inteins is published at neb.com/inteins.html.

Inteins function as protein introns and are excised out of a protein while the remaining flaking regions (exteins) are joined by a peptide bond. Split inteins systems join two polypeptides without leaving a scar. In terms of split site selection particular attention is given to split sites which are surface exposed to lessen any steric hindrance during protein splicing. Thus, a functional reverse transcriptase and sequence-specific nuclease fusion protein can be reconstituted from two or more separate polypeptides by using a split-intein protein splicing strategy by respectively fusing dipartite domains that interact with each other on two ends of the two or more separate polypeptides desired to be joined.

3. pegRNA

The systems and methods disclosed herein include a spacer sequence and an extension sequence comprising a primer binding sequence (PBS) and a reverse transcriptase template (RTT) sequence, or one or more nucleic acids encoding thereof. In some embodiments, each of the spacer sequence, PBS, and RTT sequence are provided as a single prime editing guide RNA (pegRNA), or a nucleic acid encoding thereof. The spacer sequence directs the nuclease to bind to a DNA molecule having complementarity with the pegRNA, the PBS hybridizes with the bases upstream of the nuclease generated nick, and the RTT encodes the information of the intended edits and directs reverse transcription.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization.

The spacer sequence and the extension sequence may be selected from any of the sequences disclosed herein or fragments thereof which lack one or more nucleotides from the 5′ and/or 3′ end. In some embodiments, the spacer sequence or the extension sequence comprises one, two, three, four, five, six, seven, eight, nine, or ten substitutions as compared to the sequences disclosed herein. In some embodiments, the spacer sequence or the extension sequence comprises one, two, three, four, five, six, seven, eight, nine, or ten additional nucleotides on the 5′ and/or 3′ end as compared to the sequences disclosed herein. The spacer sequence and the extension sequence may be optimized by the described additions or substitutions for use in a variety of cell types and methods.

The pegRNAs may comprise additional structural elements or sequences including a gRNA scaffold responsible for Cas9 binding, a transcription termination sequence that the 3′ end of the molecule, and mutations or structural motifs that increase editing efficiency or enhance RNA stability or prevent RNA degradation. For example, the pegRNA may further comprise: a triple helix forming sequence (e.g., triple helix terminators from a long non-coding RNAs (lncRNAs), e.g., metastasis-associated lung adenocarcinoma transcript 1 (MALATI)); a tRNA-like sequence; a pseudoknot (e.g., a modified prequcosine 1-1 riboswitch aptamer, (evopreQ1) or the frameshifting pseudoknot from Moloney murine leukemia virus (MMLV)); and silent mutations near the intended edit (e.g., less than 10 bp away). See, for example, Nelson, et al. Nat Biotechnol. 2022 March; 40 (3): 402-410, Chen, et al., Cell. 2021 Oct. 28; 184 (22): 5635-5652.e29, International Patent Publication No. WO2022067130, each of which is incorporated herein by reference in its entirety.

The additional structural elements or sequences may be present at any location in the pegRNA which does not interfere with the function of the spacer sequence, primer binding sequence (PBS), and a reverse transcriptase template (RTT) sequence. In some embodiments, the additional structural elements or sequences are at the 3′ end of the pegRNA.

4. Nicking Guide RNA (ngRNA)

In some embodiments, the systems and methods comprise a nicking guide RNA (ngRNA) that complexes with the sequence-specific nuclease and introduces a nick in the non-edited DNA stand. In certain embodiments, the nick induced by using the ngRNA is on the opposite strand as the initial nick. In certain embodiments, the nick induced by using the ngRNA is on the same strand as the initial nick. Thus, the ngRNA sequence may target the same or different strand as the spacer sequence.

The ngRNA may be selected from any of the sequences disclosed herein or fragments thereof which lack one or more nucleotides from the 5′ and/or 3′ end. In some embodiments, the ngRNA comprises one, two, three, four, five, six, seven, eight, nine, or ten substitutions as compared to the sequences disclosed herein. In some embodiments, the ngRNA comprises one, two, three, four, five, six, seven, eight, nine, or ten additional nucleotides on the 5′ and/or 3′ end as compared to the sequences disclosed herein.

The systems and methods may further include an engineered DNA mismatch repair (MMR)-inhibitor (e.g., protein or silencing RNA), or a nucleic acid encoding thereof. Sec Chen, et al., Cell. 2021 Oct. 28; 184 (22): 5635-5652.e29, incorporated herein by reference.

Nucleic acids of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EFla (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, mycoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible expression can be accomplished by placing the nucleic acid encoding such a molecule under the control of an inducible promoter/regulatory sequence. Promoters that are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The present disclosure also provides for vectors containing the nucleic acids and cells containing the nucleic acids or vectors, thereof. The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

The vectors of the present disclosure may direct the expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. In some embodiments, the promoter is specific to retinal cells. In some embodiments, the promoter directs expression in rod and/or cone photoreceptor cells. Suitable retinal, rod, and/or conc photoreceptor cell promoters are described elsewhere herein.

Additionally, the vector may contain, for example, some or all of the following: a selectable marker gene for selection of stable or transient transfectants in host cells; transcription termination and RNA processing signals; 5′- and 3′-untranslated regions; internal ribosome binding sites (IRESes), versatile multiple cloning sites; and reporter gene for assessing expression of the chimeric receptor. Suitable vectors and methods for producing vectors containing transgenes are well known and available in the art. Selectable markers include chloramphenicol resistance, tetracycline resistance, spectinomycin resistance, neomycin, streptomycin resistance, crythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into a cell, the vectors may be maintained as an autonomously replicating sequence or extrachromosomal element or may be integrated into host DNA.

Thus, the disclosure further provides for cells comprising a system for modifying the peripherin-2 gene, or one or more nucleic acids or vectors encoding thereof, as disclosed herein.

Conventional viral and non-viral based gene transfer methods can be used to introduce the nucleic acids into cells, tissues, or a subject. Such methods can be used to administer the nucleic acids to cells in culture, or in a host organism. Non-viral vector delivery systems include DNA plasmids, cosmids, RNA (e.g., a transcript of a vector described herein), and a nucleic acid complexed with a delivery vehicle.

Viral vector delivery systems include DNA and RNA viruses, which have either episomal or integrated genomes after delivery to the cell. A variety of viral constructs may be used to deliver the present nucleic acids to the cells, tissues and/or a subject. Viral vectors include, for example, retroviral, lentiviral, adenoviral, adeno-associated, baculoviral, and herpes simplex viral vectors. Nonlimiting examples of such recombinant viruses include recombinant adeno-associated virus (AAV), recombinant adenoviruses, recombinant lentiviruses, recombinant retroviruses, recombinant herpes simplex viruses, recombinant baculoviruses, recombinant poxviruses, phages, etc. The present disclosure provides vectors capable of integration in the host genome, such as retrovirus or lentivirus. Sec, e.g., Ausubel et al., Current Protocols in Molecular Biology, John Wiley & Sons, New York, 1989; Kay, M. A., et al., 2001 Nat. Medic. 7 (1): 33-40; and Walther W. and Stein U., 2000 Drugs, 60 (2): 249-71, incorporated herein by reference.

Vectors according to the present disclosure can be transformed, transfected, or otherwise introduced into a wide variety of host cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Methods of delivering vectors to cells may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110 (6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1:27) and Ibraheem et al. (Int J Pharm. 2014 Jan. 1; 459 (1-2): 70-83), incorporated herein by reference.

As such, the disclosure provides an isolated cell comprising the vector(s) or nucleic acid(s) disclosed herein. Preferred cells are those that can be easily and reliably grown, have reasonably fast growth rates, have well characterized expression systems, and can be transformed or transfected casily and efficiently. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Envinia. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Exemplary insect cells include Sf-9 and HIS (Invitrogen, Carlsbad, Calif.) and are described in, for example, Kitts et al., Biotechniques, 14:810-817 (1993); Lucklow, Curr. Opin. Biotechnol., 4:564-572 (1993); and Lucklow et al., J. Virol., 67:4566-4579 (1993), incorporated herein by reference. A number of suitable mammalian and human host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97:4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate, rodent, and human cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, HEK, A549, HepG2, mouse L-929 cells, and BHK or HaK hamster cell lines. Methods for selecting suitable mammalian cells and methods for transformation, culture, amplification, screening, and purification of cells are known in the art.

In some embodiments, the cell is a eukaryotic cell. In some embodiments, the cell is a mammalian cell. In some embodiments, the cell is a human cell. In some embodiments, the cell is in vitro. In some embodiments, the cell is ex vivo. In some embodiments, the cell is in vivo and delivery to the cell comprises administration to a subject.

### Methods of Treating a Disease or Disorder

Also disclosed herein are methods for treating or preventing a disease or disorder in a subject caused or mitigated by mutations in the peripherin-2 gene. In some embodiments, the subject has c.828 splice site mutations in the peripherin-2 gene. In some embodiments, the subject has a +1, +2 and +3 c.828 mutations in the peripherin-2 gene. In some embodiments, the subject has a peripherin-2 gene with any or all of the following mutations c.828+3A>T, c.828+1G>A, c.828+1G>T, and c.828+2T>C.

The methods comprise administering to a subject: a sequence-specific nuclease, or a nucleic acid encoding thereof; an RNA-dependent DNA polymerase, or a nucleic acid encoding thereof; one or more RNA polynucleotides comprising a spacer sequence and an extension sequence comprising a primer binding sequence (PBS) and a reverse transcriptase template (RTT) sequence, or one or more nucleic acids encoding thereof; and optionally, a nicking guide RNA (ngRNA), or a nucleic acid encoding thereof. The systems and components disclosed herein are applicable to the methods for treatment and prevention of a disease or disorder.

In some embodiments, the disease or disorder is an ocular disease or disorder. In some embodiments, the disease or disorder is a retinal disease or disorder. In some embodiments, the disease or disorder comprises retinal degeneration, retinitis pigmentosa, macular degeneration, macular dystrophy, fundus flavimaculatus-like dystrophy, central areolar choroidal dystrophy, cone-rod dystrophy, or a combination thereof.

In some embodiments, the systems or components thereof are configured for delivery to retinal cells. In some embodiments, the system is configured for delivery to rod and cone photoreceptor cells. For example, in some embodiments, the nucleic acids encoding the components may comprise a retinal cell (e.g., rod and/or cone photoreceptor cell) promoter which directs expression of the components in the retinal cells. Suitable retinal, rod, and/or cone photoreceptor cell promoters include, but are not limited to: 770En_454P (hGRM6), a human GRM6 gene-derived, short promoter; promoters based on the 2.1-kb human L-opsin promoter (pR2.1); promoter derived from the rhodopsin kinase (RK) gene; promoter derived from the rhodopsin gene; a promoter derived from the Nrl gene; murine rhodopsin promoter (mOP); G-protein-coupled receptor protein kinase 1 (GRK1) promoter; retinol-binding protein 3, interstitial (RBP3) promoter; RPE65 promoter; human inter-photoreceptor retinoid binding protein/retinol-binding protein 3 (IRBP) promoter; and retinaldehyde binding protein 1 (RLBP1) promoter. Additionally, or alternatively, the systems or components are configured for administration to the eye and/or retina, rather than systemic administration.

Administration may be through any suitable mode of administration, including but not limited to: intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmucosal, topical, and inhalation. In some embodiments, the systems or components are delivered to the tissue(s) of interest. Such delivery may be either via a single dose, or multiple doses.

In some embodiments, an effective amount of the components of the systems, methods or compositions as described can be administered. As used herein the term “effective amount” may be used interchangeably with the term “therapeutically effective amount” and refers to that quantity that is sufficient to result in a desired activity upon administration to a subject in need thereof. Within the context of the present disclosure, the term “effective amount” refers to that quantity of the components of the system such that successful modification of the peripherin-2 gene is achieved.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

### EXAMPLES

The following are examples of the present invention and are not to be construed as limiting.

### Example 1

### PRPH2 Disease Mutation Knock-ins

Mutations in PRPH2 lead to a spectrum of inherited retinal diseases (IRDs). A 67-year-old RP patient with the splice site mutation c.828+1 G>A in PRPH2 was identified. The patient was examined at the Edward S. Harkness Eye Institute at Columbia University Medical Center (New York, NY). The study was conducted under Columbia University Institutional Review Board approval (protocol IRB AAAF1849) and all procedures were performed according to the tenets of the Declaration of Helsinki. Informed consent was gathered according to IRB AAAF1849. The patient's best corrected visual acuity was measured before administration of topical tropicamide (1%) and phenylephrine hydrochloride (2.5%) for dilation. Near-infrared autofluorescence (NIR-AF) (787 nm excitation, 830 nm emission, 30 degrees×30 degrees) was performed using a Spectralis HRA2 (Heidelberg Engineering, Heidelberg, Germany), and wide-angle color fundus photography was performed using an Optos 200Tx unit (Optos; PLC, Dunfermline, United Kingdom). Lidocaine was administered to a small region of the skin on the lower back, and a biopsy punch (Mckesson, Virginia) was used to perform the skin biopsy.

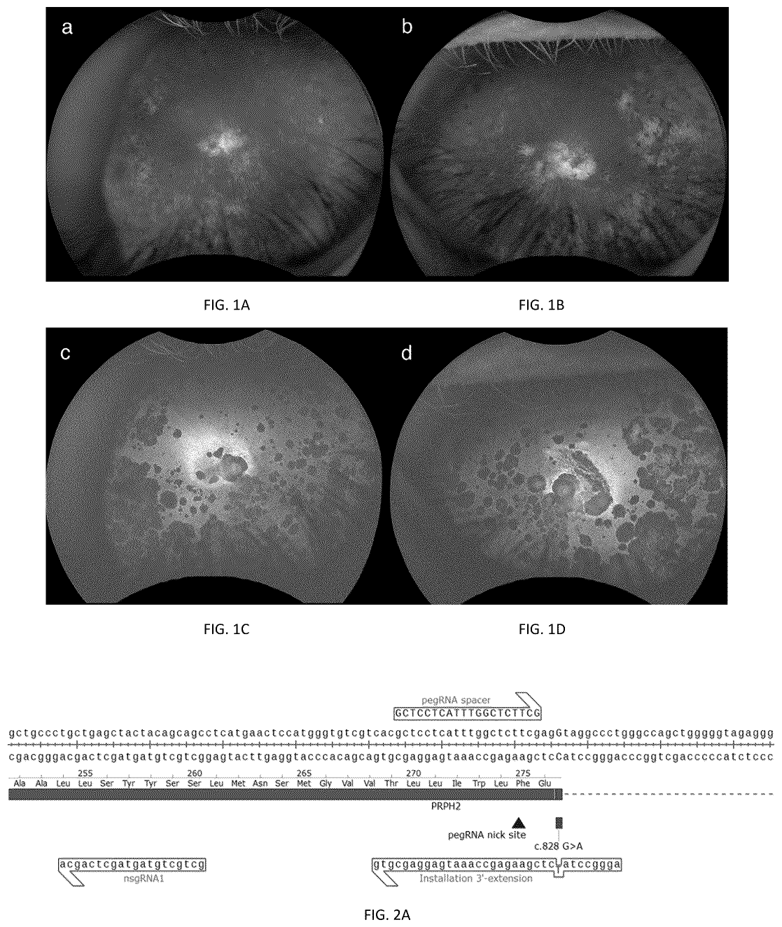

Color fundus photographs of the right and left eye demonstrated widespread RPE atrophy with a central area of macular atrophy sparing the fovea. There was sparse intraretinal pigment migration OS>OD in the inferotemporal periphery (FIGS. 1A and 1B). Fundus autofluorescence imaging of the right and left eye, respectively, reveal scattered hypoautofluorescence atrophic lesions OS>OD extending throughout the periphery with relative sparing of the superior retina (FIGS. 1C and 1D).

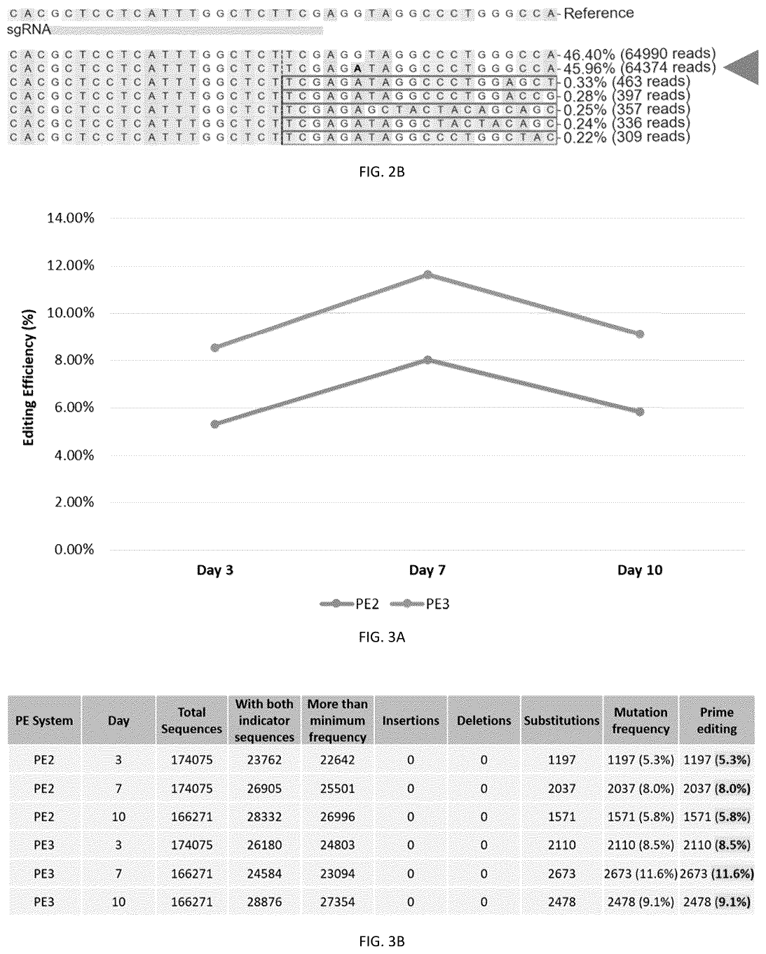

Induced pluripotent stem cell (iPSC)-derived retinal organoids are sensitive, quantitative, and scalable phenotypic assays and have been used to model several IRDs. A PE3 strategy was developed for the installation of the c.828+1 G>A in PRPH2 mutation. The method resulted in efficient editing of 45.96% in HEK293 cells (FIGS. 2A and 2B). Further, a PE2 vs PE3 strategy was compared for the installation of the c.828+1 G>A in PRPH2 mutation over 10 days (FIGS. 3A and 3B). Over the three time points analyzed the PE3 strategy was always more efficient than PE2.

Cloning and Plasmid Constructs A modified version of the Cas9 nickase-reverse transcriptase plasmid (pCMV-PE2, Addgene #132775) was used, pCMV-PE (V4), as previously described (Tsai et al., 2023 Adv Exp Med Biol, in press: In Retinal Degenerative Diseases XIX: Mechanisms and Experimental Therapy). The pegRNA was cloned into the pU6-pegRNA-GG-Vector (Addgene, #132777) and nicking guide RNA (ngRNA) was cloned into the pU6-spacer-acceptor, both using BsaI Golden Gate assembly (NEB), as described previously (Anzalone et al. 2019. Nature. 576:149-157; Tsai et al., (2023) In: Tsang, S. H., Quinn, P. M. (eds) Retinitis Pigmentosa. Methods in Molecular Biology, vol 2560). Oligos were ordered from Integrated DNA Technologies (IDT). pU6-Sp-pcgRNA-PRPH2-828+1G>A-sub: pegRNA spacer, top strand oligonucleotide: 5′-CACCGCTCCTCATTTGGCTCTTCGGTTTC-3′ (SEQ ID NO: 1); bottom strand oligonucleotide: 5′-CTCTGAAACCGAAGAGCCAAATGAGGAGC-3′ (SEQ ID NO: 2) and Installation 3′-extension, top strand oligonucleotide: 5′-GTCCAGGGCCTATCTCGAAGAGCCAAATG-3′ (SEQ ID NO: 3); bottom strand oligonucleotide: 5′-AAAACATTTGGCTCTTCGAGATAGGCCCT-3′ (SEQ ID NO: 4). pU6-ngRNA-PRPH2-828+1G>A-sub: ngRNA spacer, top strand oligonucleotide: 5′-CACCGCTGCTGTAGTAGCTCAGCA-3′ (SEQ ID NO: 5); bottom strand oligonucleotide:

(SEQ ID NO: 6)
5′-AAACTGCTGAGCTACTACAGCAGC-3′.

Cell Culture and Transfection To test editing efficiency of the prime editing, the plasmids were transfected into HEK293 cells. The HEK293 cells were seeded one day before at 50000 cells/well in 24 well plates. On the next day, the medium was refreshed with 500 ul complete medium. For the transfection of PE3, the plasmid constitution is 750 ng: 250 ng: 83 ng of CMV-PE-V4: U6-pegRNA: U6-ngRNA. For PE2, only CMV-PE-V4 and U6-pegRNA were added. Lipofectamine 2000 (Thermo Fisher) was mixed at 1:1 mass ratio with plasmid DNA. The cells were then collected after 72 hours post transfection for DNA extraction and analysis. To determine the time course of prime editing, the same number of cells were seeded in 6 well plate instead and the same amount of plasmid DNA/lipofectamine mixture was used to transfect the cells. The cells were collected at 72 hours, 168 hours, and 240 hours post transfection.

DNA extraction The cells were detached from the wells by trypsin. The cells from each well were washed with DPBS (without Ca2+ and Mg2+) and re-suspended with 50 ul DPBS. The cells were then incubated at 95° C. for 20 mins. Subsequently, after cooling, 4 ul of 20 mg/ml Proteinase K (Promega) was added to each sample. The samples were then incubated at 56° C. for one hour followed by a 30 min incubation at 95° C. to stop the proteinase K digestion. This crude extract of DNA is then ready for PCR purpose.

Analysis of Prime Editing Efficiency For the determination of editing efficiency, the PRPH2 c.828+1 locus (Forward Primer: 5′-caccagacggaggagctca-3′ (SEQ ID NO: 7), Reverse Primer: 5′-gagggaggcatgctctcca-3′ (SEQ ID NO: 8)) were amplified using primers with Illumina adaptors. The amplicon was submitted to Genewiz for the Amplicon EZ service. The analysis of the sequencing data was determined using CRISPResso2 (crispresso.pinellolab.partners.org/submission) or RGEN tools (rgenome.net/pe-analyzer/#!). The reads of the amplicons less than 0.1% of the total frequency were excluded for analysis.

c.828+2 and +3 Knock-in Using similar methods described above for the mutation c.828+1 G>A installation, pegRNA and nicking guide RNA (ngRNA) were designed as shown below.

### 3′ Extensions:

c.828+2
(SEQ ID NO: 9)
agggcctGcctcgaagagccaaatgag
c.828+3
(SEQ ID NO: 10)
agggccAacctcgaagagccaaatgagg

Spacer for ngRNA:

sg199
(SEQ ID NO: 11)
gctgctgtagtagctcagca
sg213
(SEQ ID NO: 12)
gaggagcgtgacgacaccca

### Example 2

### Prime Editing Correction for the c.828+1, +2 and +3 PRPH2 Mutations

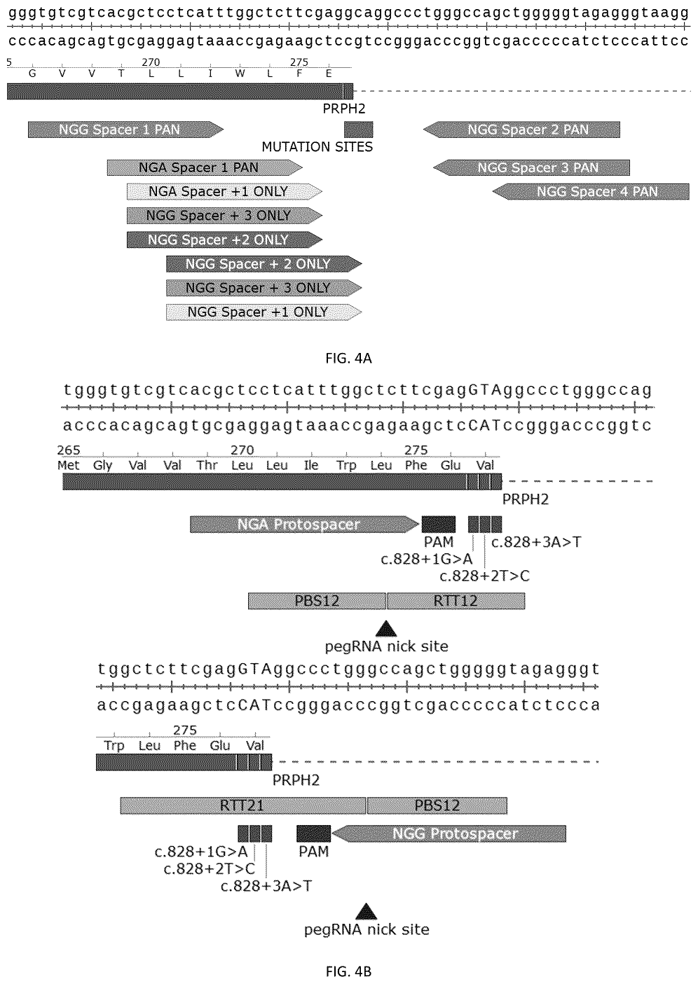

FIG. 4 is a schematic showing primer editing strategies for each PRPH2 mutation individually as well as a PAN strategy which targets all of the mutations. The mutation specific approaches involve spacers and PAMs that incorporate the patient specific mutations and so are unique to each mutation. The PAN approaches facility editing of all patient mutations, spacer and PAMs avoid incorporation of patient mutations. Thus, for the individual +1, +2 or +3 designs spacers and PAMs (e.g., NGA and NGG) will vary based on the sequence of the mutation: c.828+1G>A, c.828+2T>C and c.828+3A>T. PAMless spacer designs, for use with Cas proteins having relaxed PAM requirements, are also possible.

Correction of c.828+1G>A NGA PAM prime editing

### Spacer Sequence:

sg198
(SEQ ID NO: 24)
GCTCCTCATTTGGCTCTTCG

### 3′-Extensions:

| | | |
|---|---|---|
| RDS c.828-P12-R16 | ccagggccTaCctc | SEQ ID NO: 13 |
| | gaagagccaaatga | |
| RDS c.828-P11-R16 | ccagggccTaCctc | SEQ ID NO: 14 |
| | gaagagccaaatg | |
| RDS c.828-P10-R16 | ccagggccTaCctc | SEQ ID NO: 15 |
| | gaagagccaaat | |
| RDS c.828-P12-R12 | ggccTaCctcgaag | SEQ ID NO: 16 |
| | agccaaatga | |
| RDS c.828-P11-R12 | ggccTaCctcgaag | SEQ ID NO: 17 |
| | agccaaatg | |
| RDS c.828-P10-R12 | ggccTaCctcgaag | SEQ ID NO: 18 |
| | agccaaat | |

ngRNAs (Nick) Spacer Sequences:

| | | |
|---|---|---|
| Nick1 | gcagccacgcacccacaggt | SEQ ID NO: 19 |
| Nick2 | gacgacacccatggagttca | SEQ ID NO: 20 |
| Nick3 | gagctggcccagggcctaTct | SEQ ID NO: 21 |
| Nick4 | gcattagacccaaatgggacc | SEQ ID NO: 22 |
| Nick5 | gtgaaggctgtttccaaagag | SEQ ID NO: 23 |

### Correction of c.828+1G>A NGG PAM Prime Editing

### Spacer Sequence:

sg209
(SEQ ID NO: 25)
ctcatttggctcttcgagGt

### 3′-Extensions:

| | | |
|---|---|---|
| RDS c.828-P12-R15 | CAGGGCCTACCTCG | SEQ ID NO: 26 |
| | AAGAGCCAAATGA | |
| RDS c.828-P11-R15 | CAGGGCCTACCTCG | SEQ ID NO: 27 |
| | AAGAGCCAAATG | |
| RDS c.828-P10-R15 | CAGGGCCTACCTCG | SEQ ID NO: 28 |
| | AAGAGCCAAAT | |
| RDS c.828-P12-R14 | AGGGCCTACCTCGA | SEQ ID NO: 29 |
| | AGAGCCAAATGA | |
| RDS c.828-P11-R14 | AGGGCCTACCTCGA | SEQ ID NO: 30 |
| | AGAGCCAAATG | |
| RDS c.828-P10-R14 | AGGGCCTACCTCGA | SEQ ID NO: 31 |
| | AGAGCCAAAT | |

ngRNAs (Nick) Spacer Sequences:

| | | |
|---|---|---|
| Nick1 | gcagccacgcacccacaggt | SEQ ID NO: 32 |
| Nick2 | gacgacacccatggagttca | SEQ ID NO: 33 |
| Nick3 | gagctggcccagggcctaTct | SEQ ID NO: 34 |
| Nick4 | gcattagacccaaatgggacc | SEQ ID NO: 35 |
| Nick5 | gtgaaggctgtttccaaagag | SEQ ID NO: 36 |
| Nick6 | ggaggcatgctctccaagcc | SEQ ID NO: 37 |

### Correction of c.828+3A>T NGG PAM Prime Editing

### Spacer Sequences:

sg198
(SEQ ID NO: 38)
GCTCCTCATTTGGCTCTTCG
sg209
(SEQ ID NO: 39)
GctcatttggctcttcgagGt

### 3′-Extensions:

| | | |
|---|---|---|
| PBS12-RTT14 (A) | agggccTaCctcgaag | SEQ ID NO: 40 |
| | agccaaatga | |
| PBS12-RTT19 (A) | ggcccagggccTaCct | SEQ ID NO: 41 |
| | cgaagagccaaatga | |
| PBS16-RTT14 (A) | agggccTaCctcgaag | SEQ ID NO: 42 |
| | agccaaatgaggag | |
| PBS12-RTT14 (B) | gcccagggccTaCctc | SEQ ID NO: 43 |
| | gaagagccaa | |
| PBS12-RTT19 (B) | agctggcccagggccT | SEQ ID NO: 44 |
| | aCctcgaagagccaa | |
| PBS16-RTT14 (B) | gcccagggccTaCctc | SEQ ID NO: 45 |
| | gaagagccaaatga | |

ngRNAs (Nick) Spacer Sequences:

| | | |
|---|---|---|
| sg199 | GCTGCTGTAGTAGCTCAGCA | SEQ ID NO: 46 |
| sg210 | gccctgcagccacgcacccac | SEQ ID NO: 47 |
| sg211 | gcgacacccatggagttcatg | SEQ ID NO: 48 |
| sg212 | gccttaccctctacccccagc | SEQ ID NO: 49 |
| sg213 | gaggagcgtgacgacaccca | SEQ ID NO: 50 |
| sg214 | gttagacccaaatgggaccgg | SEQ ID NO: 51 |
| sg215 | gacaggttgagctcctccgtc | SEQ ID NO: 52 |
| sg216 | ggaggcatgctctccaagcc | SEQ ID NO: 53 |
| sg217 | gcgcccccattagacccaaat | SEQ ID NO: 54 |
| sg218 | gcTaCctcgaagagccaaatg | SEQ ID NO: 55 |
| sg229 | gctctacccccagctggccca | SEQ ID NO: 56 |
| sg243 | ggctgctgtagtagctcagc | SEQ ID NO: 57 |

PAN Correction of c.828+1, +2, and +3 Mutations with Prime Editing

### 1. NGG PAM Prime Editor

### Spacer Sequences:

sg229
(SEQ ID NO: 58)
gctctacccccagctggccca

### 3′-Extensions:

| | | |
|---|---|---|
| PBS14-RTT21 | gctcttcgagGtAggccctg | SEQ ID NO: 59 |
| | ggccagctgggggta | |
| PBS14-RTT16 | tcgagGtAggccctgggcca | SEQ ID NO: 60 |
| | gctgggggta | |

ngRNAs (Nick) Spacer Sequences:

| | | |
|---|---|---|
| sg230 | gacggaggagctcaacctgt | SEQ ID NO: 61 |
| sg231 | gagctcaacctgtgggtgcg | SEQ ID NO: 62 |
| sg232 | gcctgtgggtgcgtggctgca | SEQ ID NO: 63 |
| sg233 | gcagcagcctcatgaactcca | SEQ ID NO: 64 |
| sg234 | gtgtcgtcacgctcctcatt | SEQ ID NO: 65 |
| sg235 | gctgggccagctgggggtaga | SEQ ID NO: 66 |
| sg236 | gccagctgggggtagagggta | SEQ ID NO: 67 |
| sg237 | gtagagggtaaggagagcctc | SEQ ID NO: 68 |
| sg238 | gagagcctccggtcccattt | SEQ ID NO: 69 |
| sg239 | gcggtcccatttgggtctaat | SEQ ID NO: 70 |
| sg240 | gccatttgggtctaatggggg | SEQ ID NO: 71 |
| sg241 | ggtctaatgggggcggagcc | SEQ ID NO: 72 |
| sg242 | gaatgggggcggagccaggct | SEQ ID NO: 73 |

### 2. NGA PAM Prime Editor

### Spacer Sequences:

sg219
(SEQ ID NO: 74)
gacgctcctcatttggctctt

### 3′-Extensions:

| | | |
|---|---|---|
| PBS12-RTT20 | gcccagggccTaCctcgaagagccaaatgagg | SEQ ID NO: 75 |
| PBS12-RTT14 | tggccTaCctcgaagagccaaatgagg | SEQ ID NO: 76 |
| PBS10-RTT13 | gccTaCctcgaagagccaaatga | SEQ ID NO: 77 |
| PBS10-RTT16 | agggccTaCctcgaagagccaaatga | SEQ ID NO: 78 |
| PBS10-RTT20 | gcccagggccTaCctcgaagagccaaatga | SEQ ID NO: 79 |
| PBS10-RTT22 | tggcccagggccTaCctcgaagagccaaatga | SEQ ID NO: 80 |
| PBS10-RTT25 | agctggcccagggccTaCctcgaagagccaaatga | SEQ ID NO: 81 |
| PBS11-RTT13 | gccTaCctcgaagagccaaatgag | SEQ ID NO: 82 |
| PBS11-RTT16 | agggccTaCctcgaagagccaaatgag | SEQ ID NO: 83 |
| PBS11-RTT20 | gcccagggccTaCctcgaagagccaaatgag | SEQ ID NO: 84 |
| PBS11-RTT22 | tggcccagggccTaCctcgaagagccaaatgag | SEQ ID NO: 85 |
| PBS11-RTT25 | agctggcccagggccTaCctcgaagagccaaatgag | SEQ ID NO: 86 |
| PBS12-RTT13 | gccTaCctcgaagagccaaatgagg | SEQ ID NO: 87 |
| PBS12-RTT16 | agggccTaCctcgaagagccaaatgagg | SEQ ID NO: 88 |
| PBS12-RTT20 | gcccagggccTaCctcgaagagccaaatgagg | SEQ ID NO: 89 |
| PBS12-RTT22 | tggcccagggccTaCctcgaagagccaaatgagg | SEQ ID NO: 90 |
| PBS12-RTT25 | agctggcccagggccTaCctcgaagagccaaatgagg | SEQ ID NO: 91 |
| PBS13-RTT13 | gccTaCctcgaagagccaaatgagga | SEQ ID NO: 92 |
| PBS13-RTT16 | agggccTaCctcgaagagccaaatgagga | SEQ ID NO: 93 |
| PBS13-RTT20 | gcccagggccTaCctcgaagagccaaatgagga | SEQ ID NO: 94 |
| PBS13-RTT22 | tggcccagggccTaCctcgaagagccaaatgagga | SEQ ID NO: 95 |
| PBS13-RTT25 | agctggcccagggccTaCctcgaagagccaaatgagga | SEQ ID NO: 96 |
| PBS14-RTT13 | gccTaCctcgaagagccaaatgaggag | SEQ ID NO: 97 |
| PBS14-RTT16 | agggccTaCctcgaagagccaaatgaggag | SEQ ID NO: 98 |
| PBS14-RTT20 | gcccagggccTaCctcgaagagccaaatgaggag | SEQ ID NO: 99 |
| PBS14-RTT22 | tggcccagggccTaCctcgaagagccaaatgaggag | SEQ ID NO: 100 |
| PBS14-RTT25 | agctggcccagggccTaCctcgaagagccaaatgaggag | SEQ ID NO: 101 |
| PBS15-RTT13 | gccTaCctcgaagagccaaatgaggagc | SEQ ID NO: 102 |
| PBS15-RTT16 | agggccTaCctcgaagagccaaatgaggagc | SEQ ID NO: 103 |
| PBS15-RTT20 | gcccagggccTaCctcgaagagccaaatgaggagc | SEQ ID NO: 104 |
| PBS15-RTT22 | tggcccagggccTaCctcgaagagccaaatgaggagc | SEQ ID NO: 105 |
| PBS15-RTT25 | agctggcccagggccTaCctcgaagagccaaatgaggagc | SEQ ID NO: 106 |
| PBS16-RTT13 | gccTaCctcgaagagccaaatgaggagcg | SEQ ID NO: 107 |
| PBS16-RTT16 | agggccTaCctcgaagagccaaatgaggagcg | SEQ ID NO: 108 |
| PBS16-RTT20 | gcccagggccTaCctcgaagagccaaatgaggagcg | SEQ ID NO: 109 |
| PBS16-RTT22 | tggcccagggccTaCctcgaagagccaaatgaggagcg | SEQ ID NO: 110 |
| PBS16-RTT25 | agctggcccagggccTaCctcgaagagccaaatgaggagcg | SEQ ID NO: 111 |
| PBS18-RTT13 | gccTaCctcgaagagccaaatgaggagcgtg | SEQ ID NO: 112 |
| PBS18-RTT16 | agggccTaCctcgaagagccaaatgaggagcgtg | SEQ ID NO: 113 |
| PBS18-RTT20 | gcccagggccTaCctcgaagagccaaatgaggagcgtg | SEQ ID NO: 114 |
| PBS18-RTT22 | tggcccagggccTaCctcgaagagccaaatgaggagcgtg | SEQ ID NO: 115 |
| PBS18-RTT25 | agctggcccagggccTaCctcgaagagccaaatgaggagcgtg | SEQ ID NO: 116 |

ngRNAs (Nick) Spacer Sequences:

| | | |
|---|---|---|
| sg220 | gcagccacgcacccacaggt | SEQ ID NO: 117 |
| sg221 | gaggagcgtgacgacacccat | SEQ ID NO: 118 |
| sg222 | gacgacacccatggagttca | SEQ ID NO: 119 |
| sg223 | gaagagccaaatgaggagcg | SEQ ID NO: 120 |
| sg224 | gcattagacccaaatgggacc | SEQ ID NO: 121 |
| sg225 | gtgaaggctgtttccaaagag | SEQ ID NO: 122 |
| sg226 | gagcctggctccgcccccatt | SEQ ID NO: 123 |
| sg227 | gcccccattagacccaaatg | SEQ ID NO: 124 |
| sg228 | gagccaaatgaggagcgtga | SEQ ID NO: 125 |

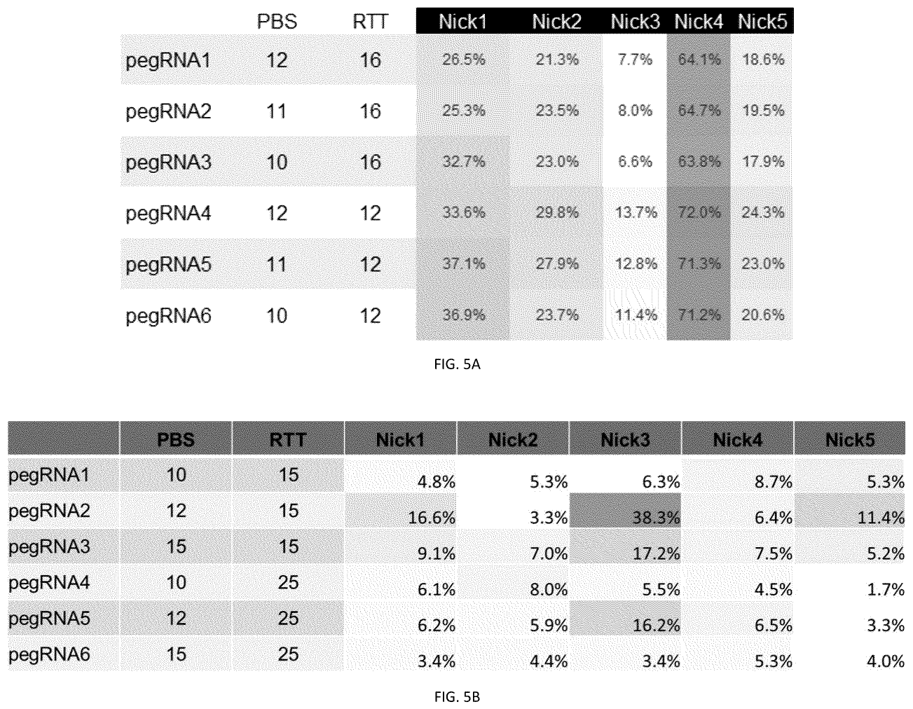

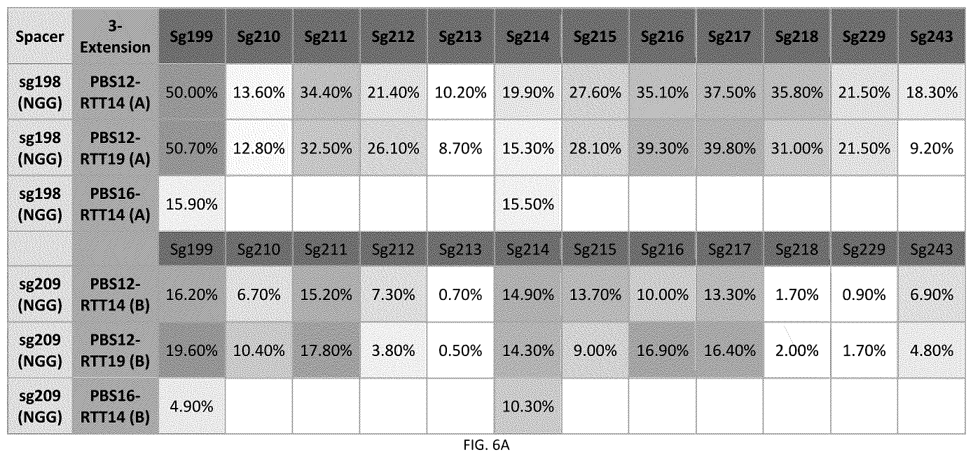

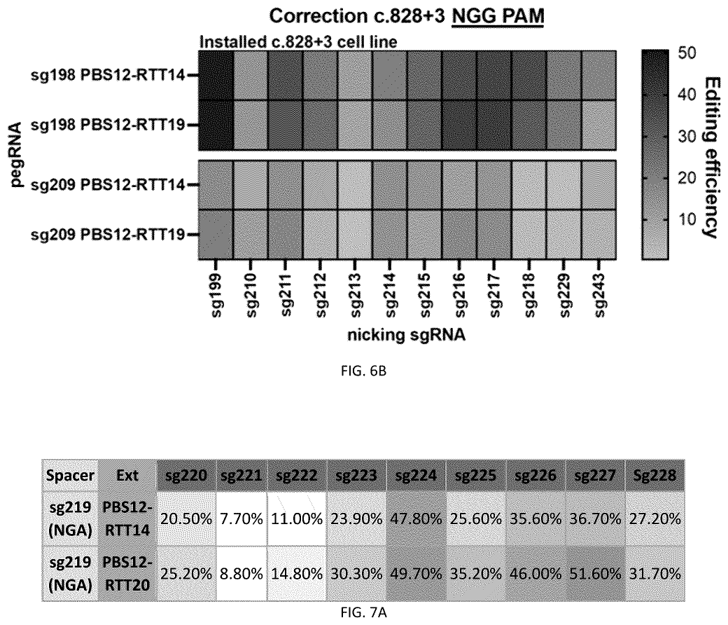

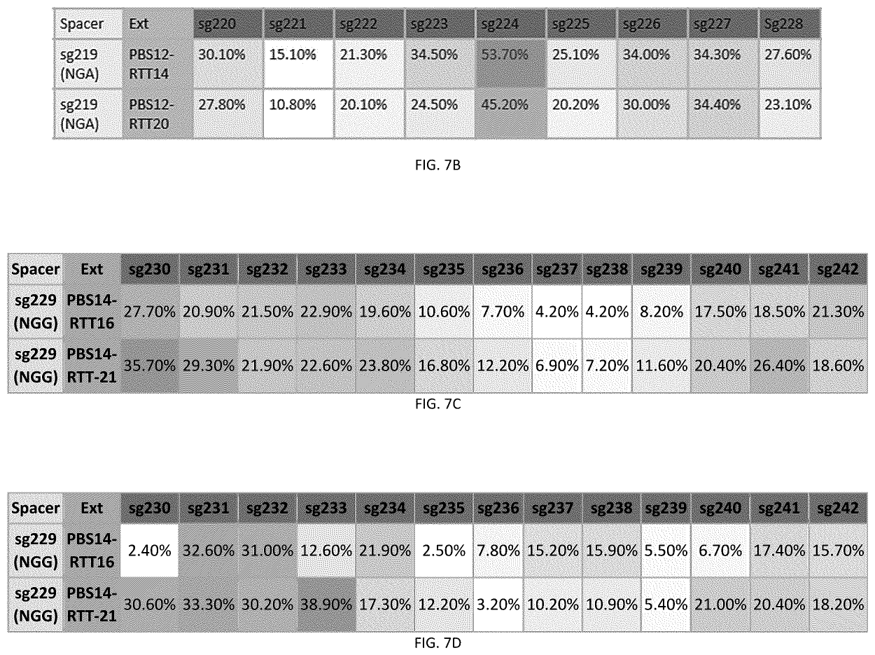

To test editing efficiency of the prime editing components, the plasmids encoding the pegRNA and nicking single guide RNA were transfected into HEK293 cells as described in Example 1. FIGS. 5A and 5B show the efficiency of c.828+1G>A NGG and NGA PAM prime editing correction, respectively. FIG. 6 shows the efficiency of correction of c.828+3A>T. FIGS. 7A-7D show the efficiency of correction of the +3 mutation (FIGS. 7A and 7C) and the +1 mutation (FIGS. 7B and 7D) for the NGA and NGG PAM prime editing strategies.

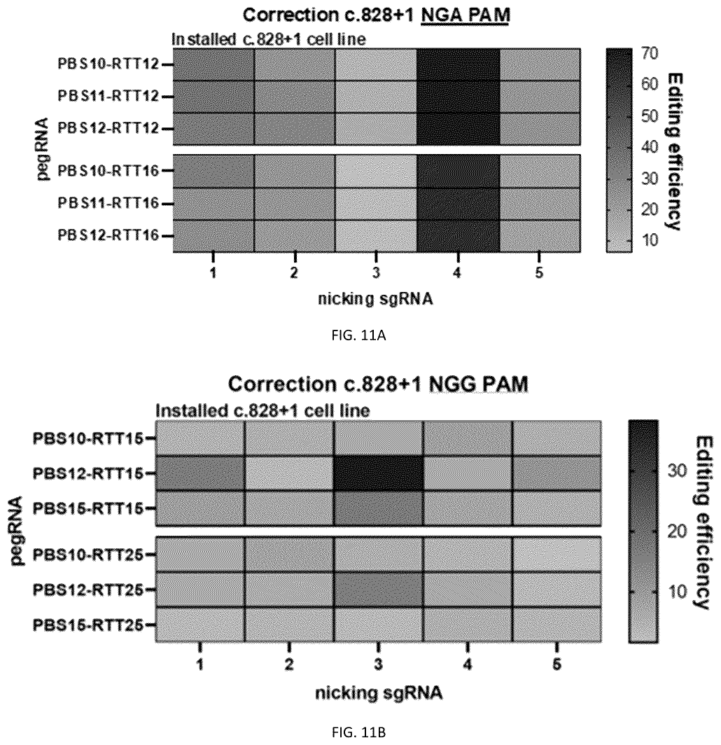

For both the c.828+1G>A (FIGS. 11A and 11B) and c.828+3A>T (FIG. 6) PRPH2 mutations mutation specific prime editing approaches were screened in their corresponding homozygous knock-in HEK293 cell lines. For the c.828+1G>A experiments 50,000 cells/well in a 24 well plate was used. For the c.828+3A>T experiments, 14,000 cells/well in a 96 well plate was used. All experiments were performed in triplicate and editing efficiency was determined by next generation sequencing (NGS). For the c.828+1G>A PRPH2 mutation an NGA (FIG. 11A) and NGG (FIG. 11B) spacer were evaluated for the pegRNA screening, using a 3′ extension with PBSs and RTTs of varying length in addition to evaluating five nicking sgRNAs (NGA and NGG, respectively). For the c.828+1G>A NGA spacer based correction (FIG. 11A) the highest editing achieved was 72% using PBS12-RTT12 and nicking sgRNA 4. For the c.828+1G>A NGG spacer based correction (FIG. 11B) the highest editing achieved was 38.3% using PBS12-RTT15 and nicking sgRNA 3. These experiments were performed in the same method and show that the NGA based spacer led to higher editing efficiencies. For the c.828+3A>T PRPH2 mutation two different mutation specific NGG spacers with 3′ extensions that had a set PBS length of 12 and either a long or short RTT in addition to evaluating 12 nicking sgRNAs were screened (FIG. 6). For the c.828+3A>T PRPH2 mutation the sg198 spacer using a 3′ extension of PBS12-RTT19 and nicking sgRNA199 had the highest editing efficiency of 50.7%. These outcomes show the feasibility of mutation specific prime editing for the correction of the c.828+1G>A and c.828+3A>T PRPH2 mutations and establish a platform for further improving editing efficiency through further refinements of PBS/RTT length and nicking sgRNA. All the experiments (mutation specific and PAN mutation) include the use of structured RNA motifs to the 3′ terminus of pegRNAs to prevent the degradation of the 3′ extension by exonucleases and improve editing efficiency.

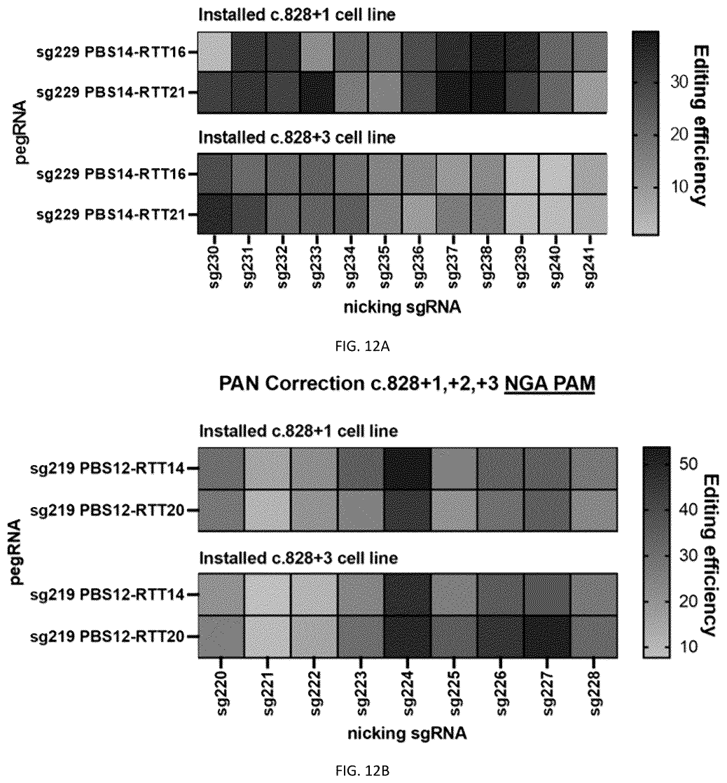

For both the c.828+1G>A and c.828+3A>T PRPH2 mutations PAN mutation prime editing approaches were screened (FIGS. 7A-7D and 12A-12B) in their corresponding homozygous knock-in HEK293 cell lines. For the c.828+1G>A and c.828+3A>T PAN mutation prime editing experiments, 14,000 cells/well in a 96 well plate was used. All experiments were performed in triplicate and editing efficiency was determined by next generation sequencing (NGS). Two pegRNAs with cither an NGG based spacer (FIGS. 7C-7D and 12A) or NGA based spacer (FIGS. 7A-7B, 12B) for PAN correction were evaluated in the knock-in lines. For the c.828+1G>A the sg229 NGG based pegRNA with the PBS14-RTT21 3′ extension in combination with the sg238 nicking sgRNA gave the highest editing efficiency, at 39.8% (FIG. 12A). However, this same combination in the c.828+3T>A line gave an editing efficiency of only 16.4%. For the c.828+3A>T line the sg229 NGG based pegRNA with the PBS14-RTT21 3′ extension in combination with the sg230 nicking sgRNA gave the highest editing efficiency, at 35.7% (FIGS. 7C and 12A). However, this same combination in the c.828+1G>A line gave an editing efficiency of 30.6% (FIG. 7D). For the c.828+1G>A line the sg219 NGA based pegRNA with the PBS12-RTT14 3′ extension in combination with the sg224 nicking sgRNA gave the highest editing efficiency, at 53.7% (FIGS. 7B and 12B). The longer 3′ extension, PBS12-RTT20, gave the second highest editing efficiency, 45.2%, in combination with the sg224 nicking sgRNA. The same combinations in the c.828+3T>A line with the sg224 nicking sgRNA gave 47.8% and 49.7%, respectively (FIGS. 7A and 12B). However, for the c.828+3A>T line the sg219 NGG based pegRNA with the PBS12-RTT20 3′ extension in combination with the sg227 nicking sgRNA gave the highest editing efficiency, at 51.6% (FIGS. 7A and 12B). However, this same combination in the c.828+1G>A line only gave an editing efficiency of 34.4%. As both, the mutation specific approach for the c.828+3A>T (FIG. 6) and the PAN mutation approaches (FIGS. 7A-7D and 12A-12B) were performed under the same method, 96 well late with 14,000 cell/well, they can be directly compared. A similarly high editing efficiency is achieved for the correction of the c.828+3A>T using a mutation specific approach (50.7%, sg198-PBS12-RTT19, nicking sgRNA199 (FIG. 6)) and the c.828+1G>A and c.828+3A>T using a PAN mutation approach (45.2% (+1) and 49.7% (+3), sg219-PBS12-RTT20, nicking sgRNA224 (FIGS. 7A-7B, 12B)). This PAN mutation approach favors the prevalent c.828+3A>T mutation while not drastically sacrificing editing efficiency in the c.828+1G>A line. Additional improvements in editing efficiency through further refinements of PBS/RTT length and nicking sgRNA strategy can further strengthen the PAN mutation approach for the c.828 splice site mutations.

### Example 3

### PRPH2 c.828 Splice Site-Specific Mutations

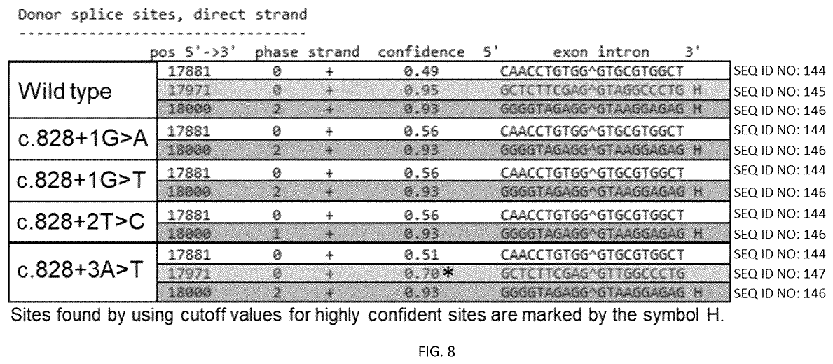

The 2^{nd} most commonly reported PRPH2 mutation is the c.828+3A>T and three rarer c.828+ (formerly annotated as IVS2+) splice site mutations have also been reported, c.828+1G>A, c.828+1G>T and c.828+2T>C (LOVD, accessed Sep. 27, 2022). The prevalence of the c.828+3A>T PRPH2 mutation is owning to a founder effect. The mutation is associated with a wide range of clinical diagnosis that fall into two clinical categories: Group 1 with mild pattern dystrophies (PD) and Group 2 with severe cone-rod dystrophy (CRD), autosomal dominant retinitis pigmentosa (adRP) and central areolar choroidal dystrophy (CACD). The PRPH2 c.828+3A>T mutation likely results in these distinct phenotypes due to a common in trans protein haplotype. Netgene2 splice site prediction program was used to evaluate the c.828+3A>T mutation finding the same predicted reduction in the activity of the canonical splice site from 95% to 70% (FIG. 8). However, for the +1 and +2 c.828 variants the activity of this canonical splice site was predicted to be completely abolished (FIG. 8). Additionally, others previously identified a cryptic splice site located 29 bp downstream in intron 2, as predicted by Netgene2 (FIG. 8), and using c.828+3A>T patient peripheral white blood cells containing illegitimate transcripts found that the mutation led to an aberrant transcript that included this predicted 29 bp of intron 2. The aberrantly spliced transcript was not found in healthy individuals.

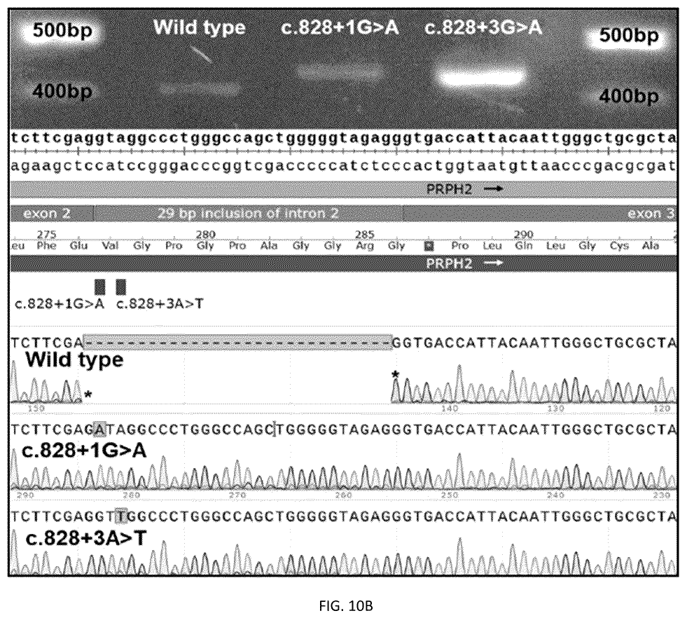

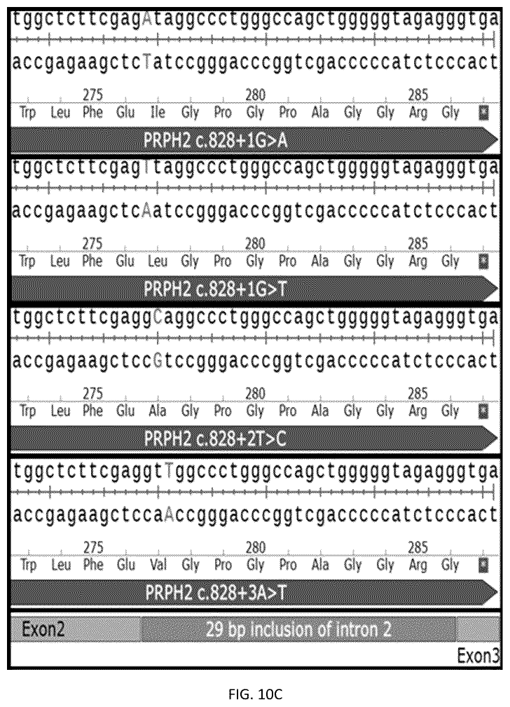

To further test this, the PRPH2 c.828 splice site HEK293T knock-in lines were used to confirm the presence of the aberrantly spliced transcript (FIG. 10B). As such, all the PRPH2 c.828 splice site mutations should lead to predicted proteins that include 10 additional amino acids after the canonical exon 2 using the 29 bp of intron 2 in addition to the first base pair of exon 3. The splicing into exon 3, the last exon of PRPH2, was predicted to introduce a premature stop codon leading to a truncated protein that should escape nonsense-mediated decay (FIG. 10C). While these findings confirm and highlight the reasoning why c.828 splice site mutations lead to inherited retinal diseases, they also provide an outcome for the evaluation of therapeutic gene editing at the transcript level.

### Example 4

### Split-Intein Prime Editing of PRPH2 c.828 Splice Site Mutations

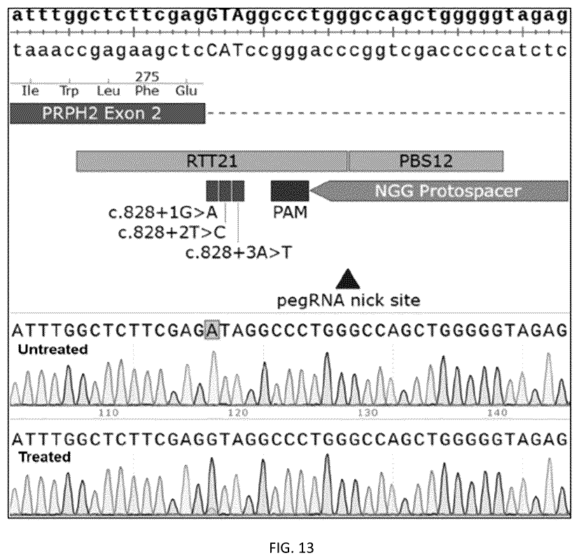

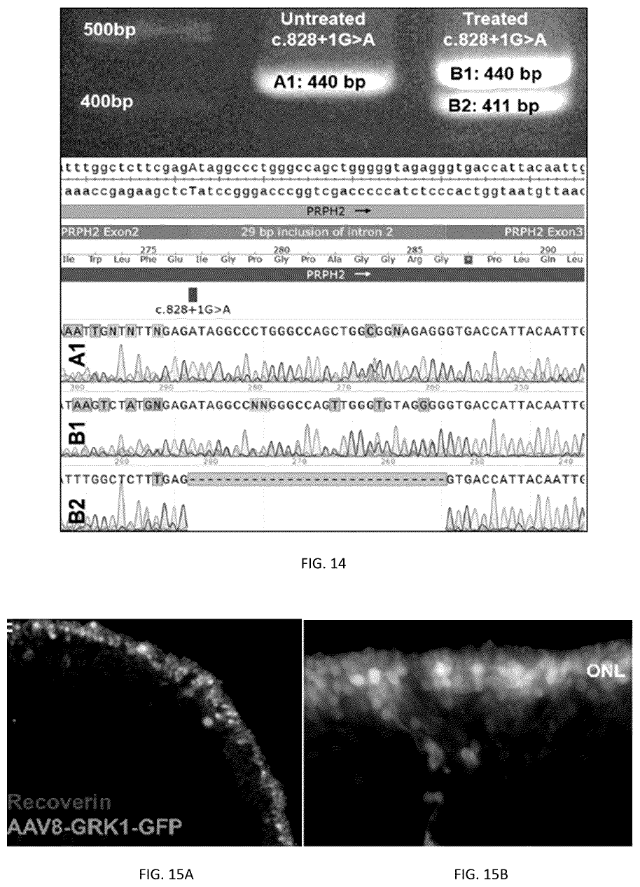

Split-intein prime editors have been used to achieve successful in vivo editing. The C-PE and N-PE AAV plasmids of the truncated MMLV reverse transcriptase prime editor (˜5.7 kb) have been shown to have comparable editing efficiency while utilizing a preferred split site for more efficient protein reconstitution (Zongliang Gao, et al., Molecular Therapy, Volume 30, Issue 9, 2022, Pages 2942-2951). This system was adapted for use with the PRPH2 c.828 splice site mutations in the described HEK293 knock-in lines and patient iPSCs, for example by using the disclosed prime editing components and by changing the promoter to a truncated CMV promoter. An N-PE insertion vector encoding the pegRNA and nsgRNA as separate components was also designed and utilized. The first nsgRNA and pegRNA for a PAN mutation correction approach for the PRPH2 c.828 splice site mutations achieved successful correction of the c.828+1G>A mutation (FIG. 13). The c.828 splice site mutations were previously found to lead to the use of a cryptic splice site causing an aberrant transcript that includes 29 bp of intron 2 of PRPH2 (FIGS. 8 and 10). In the PAN mutation split intein treated c.828+1G>A HEK293 knock-in line restoration of the wildtype PRPH2 mRNA transcript we achieved (FIG. 14). This split intein prime editor can be adapted to make PEmax NGG and NGA versions to use with pegRNAs and nicking sgRNAs disclosed above.

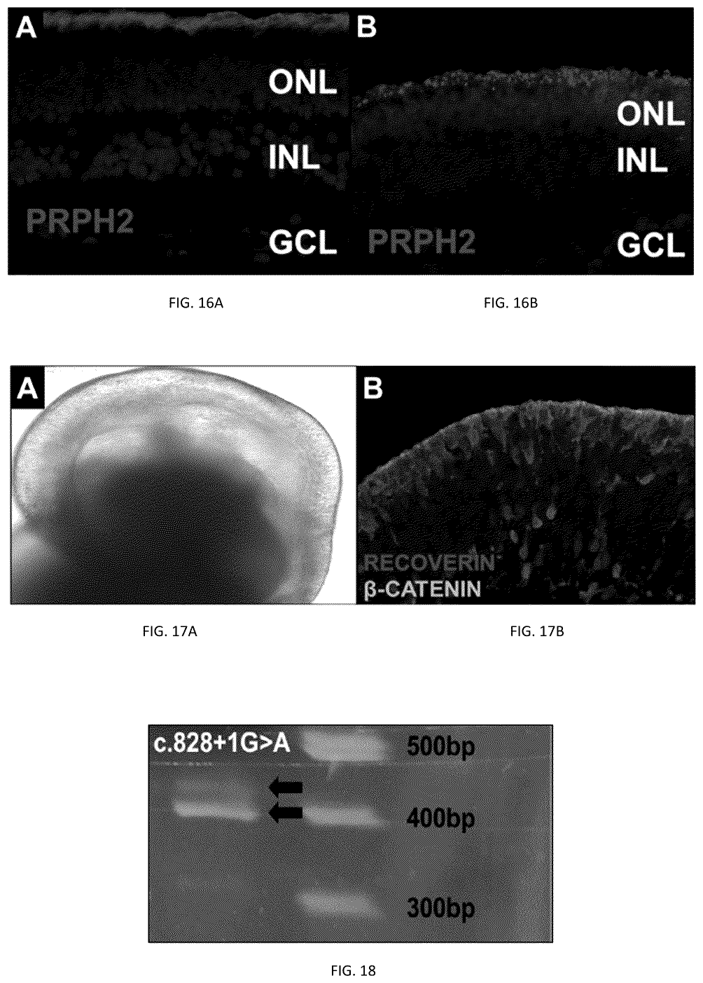

iPSC-derived retinal organoids have been successfully transduced with AAVs using various serotypes and promoter combinations and used to model inherited retinal diseases and in transgene expression assays (FIG. 15 and data not shown). PRPH2 is expressed in the photoreceptor outer segments in adult human cadaveric retina and maturated iPSC-derived retinal organoids (FIG. 16). iPSC lines have been established from one rare c.828+1G>A patient (FIG. 17) and are being generated iPSC lines from collected fibroblasts of two c.828+3A>T patients. The c.828+1G>A line forms well laminated retinal organoids and will with the two c.828+3A>T iPSC-derived retinal organoid models act as clinically relevant surrogates for testing therapeutic editing. The presence of the aberrant mRNA splicing was found in the c.828+1G>A iPSC-derived retinal organoid model (FIG. 18). These models can be used to assess restoration of canonical mRNA splicing and full length PRPH2 upon therapeutic editing.

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A system for modifying a peripherin-2 gene comprising:
a Cas protein, or a nucleic acid encoding thereof;
a reverse transcriptase, or a nucleic acid encoding thereof;
one or more RNA polynucleotides comprising a spacer sequence and an extension sequence comprising a primer binding sequence (PBS) and a reverse transcriptase template (RTT) sequence; or one or more nucleic acids encoding thereof; and
optionally, a nicking guide RNA (ngRNA), or a nucleic acid encoding thereof,
wherein the RTT sequence encodes one or more base substitutions to modify the peripherin-2 gene sequence.

2. The system of claim 1, wherein the spacer sequence and the extension sequence are contained within a single RNA polynucleotide.

3. The system of claim 1, wherein the Cas protein is Cas9 or a variant or fragment thereof.

4. The system of claim 1, wherein the Cas protein is a Cas9 nickase and/or the Cas protein comprises a Cas protein variant configured to target an expanded range of PAM sequences.

5. The system of claim 1, wherein the Cas protein and the reverse transcriptase are contained within a single fusion protein.

6. The system of claim 1, wherein the peripherin-2 gene is a mutant peripherin-2 gene comprising one or more disease-causing mutations.

7. The system of claim 1, wherein the one or more base substitutions correct one or more splice site mutations in the peripherin-2 gene.

8. The system of claim 7, wherein the one or more splice site mutations include any or all of the following splice site mutations: c.828+3A>T, c.828+1G>A, c.828+1G>T, and c.828+2T>C.

9. The system of claim 1, wherein the peripherin-2 gene is a wild-type peripherin-2 gene and wherein the one or more base substitutions install one or more splice site mutations in the peripherin-2 gene.

10. A method for modifying a peripherin-2 gene comprising contacting a DNA encoding the peripherin-2 gene with a system of claim 1.

11. The method of claim 10, wherein the peripherin-2 gene comprises one or more splice site mutations and the RTT sequence encodes one or more nucleotides to correct any one or all of the one or more splice site mutations in the peripherin-2 gene.

12. The method of claim 10, wherein the one or more splice site mutations include any or all of the following splice site mutations: c.828+3A>T, c.828+1G>A, c.828+1G>T, and c.828+2T>C.

13. A method of treating or preventing a disease or disorder in a subject in need thereof comprising administering of a system of claim 1 to the subject,
wherein the disease or disorder is caused or mitigated by mutations in peripherin-2 gene.

14. The method of claim 13, wherein the disease or disorder comprises retinal degeneration, retinitis pigmentosa, macular degeneration, macular dystrophy, fundus flavimaculatus-like dystrophy, central areolar choroidal dystrophy, cone-rod dystrophy, or a combination thereof.

15. The method of claim 13, wherein the system is configured for delivery to retinal cells.


SEQUENCE LISTING (175 sequences)

SEQ ID NO: 1 - DNA, 29 bp, synthetic construct
  source 1..29: mol_type = other DNA
caccgctcctcatttggctcttcggtttc

SEQ ID NO: 2 - DNA, 29 bp, synthetic construct
  source 1..29: mol_type = other DNA
ctctgaaaccgaagagccaaatgaggagc

SEQ ID NO: 3 - DNA, 29 bp, synthetic construct
  source 1..29: mol_type = other DNA
gtccagggcctatctcgaagagccaaatg

SEQ ID NO: 4 - DNA, 29 bp, synthetic construct
  source 1..29: mol_type = other DNA
aaaacatttggctcttcgagataggccct

SEQ ID NO: 5 - DNA, 24 bp, synthetic construct
  source 1..24: mol_type = other DNA
caccgctgctgtagtagctcagca

SEQ ID NO: 6 - DNA, 24 bp, synthetic construct
  source 1..24: mol_type = other DNA
aaactgctgagctactacagcagc

SEQ ID NO: 7 - DNA, 19 bp, synthetic construct
  source 1..19: mol_type = other DNA
caccagacggaggagctca

SEQ ID NO: 8 - DNA, 19 bp, synthetic construct
  source 1..19: mol_type = other DNA
gagggaggcatgctctcca

SEQ ID NO: 9 - DNA, 27 bp, synthetic construct
  source 1..27: mol_type = other DNA
agggcctgcctcgaagagccaaatgag

SEQ ID NO: 10 - DNA, 28 bp, synthetic construct
  source 1..28: mol_type = other DNA
agggccaacctcgaagagccaaatgagg

SEQ ID NO: 11 - DNA, 20 bp, synthetic construct
  source 1..20: mol_type = other DNA
gctgctgtagtagctcagca

SEQ ID NO: 12 - DNA, 20 bp, synthetic construct
  source 1..20: mol_type = other DNA
gaggagcgtgacgacaccca

SEQ ID NO: 13 - DNA, 28 bp, synthetic construct
  source 1..28: mol_type = other DNA
ccagggcctacctcgaagagccaaatga

SEQ ID NO: 14 - DNA, 27 bp, synthetic construct
  source 1..27: mol_type = other DNA
ccagggcctacctcgaagagccaaatg

SEQ ID NO: 15 - DNA, 26 bp, synthetic construct
  source 1..26: mol_type = other DNA
ccagggcctacctcgaagagccaaat

SEQ ID NO: 16 - DNA, 24 bp, synthetic construct
  source 1..24: mol_type = other DNA
ggcctacctcgaagagccaaatga

SEQ ID NO: 17 - DNA, 23 bp, synthetic construct
  source 1..23: mol_type = other DNA
ggcctacctcgaagagccaaatg

SEQ ID NO: 18 - DNA, 22 bp, synthetic construct
  source 1..22: mol_type = other DNA
ggcctacctcgaagagccaaat

SEQ ID NO: 19 - DNA, 20 bp, synthetic construct
  source 1..20: mol_type = other DNA
gcagccacgcacccacaggt

SEQ ID NO: 20 - DNA, 20 bp, synthetic construct
  source 1..20: mol_type = other DNA
gacgacacccatggagttca

SEQ ID NO: 21 - DNA, 21 bp, synthetic construct
  source 1..21: mol_type = other DNA
gagctggcccagggcctatct

SEQ ID NO: 22 - DNA, 21 bp, synthetic construct
  source 1..21: mol_type = other DNA
gcattagacccaaatgggacc

SEQ ID NO: 23 - DNA, 21 bp, synthetic construct
  source 1..21: mol_type = other DNA
gtgaaggctgtttccaaagag

SEQ ID NO: 24 - DNA, 20 bp, synthetic construct
  source 1..20: mol_type = other DNA
gctcctcatttggctcttcg

SEQ ID NO: 25 - DNA, 20 bp, synthetic construct
  source 1..20: mol_type = other DNA
ctcatttggctcttcgaggt

SEQ ID NO: 26 - DNA, 27 bp, synthetic construct
  source 1..27: mol_type = other DNA
cagggcctacctcgaagagccaaatga

SEQ ID NO: 27 - DNA, 26 bp, synthetic construct
  source 1..26: mol_type = other DNA
cagggcctacctcgaagagccaaatg

SEQ ID NO: 28 - DNA, 25 bp, synthetic construct
  source 1..25: mol_type = other DNA
cagggcctacctcgaagagccaaat

SEQ ID NO: 29 - DNA, 26 bp, synthetic construct
  source 1..26: mol_type = other DNA
agggcctacctcgaagagccaaatga

SEQ ID NO: 30 - DNA, 25 bp, synthetic construct
  source 1..25: mol_type = other DNA
agggcctacctcgaagagccaaatg

SEQ ID NO: 31 - DNA, 24 bp, synthetic construct
  source 1..24: mol_type = other DNA
agggcctacctcgaagagccaaat

SEQ ID NO: 32 - DNA, 20 bp, synthetic construct
  source 1..20: mol_type = other DNA
gcagccacgcacccacaggt

SEQ ID NO: 33 - DNA, 20 bp, synthetic construct
  source 1..20: mol_type = other DNA
gacgacacccatggagttca

SEQ ID NO: 34 - DNA, 21 bp, synthetic construct
  source 1..21: mol_type = other DNA
gagctggcccagggcctatct

SEQ ID NO: 35 - DNA, 21 bp, synthetic construct
  source 1..21: mol_type = other DNA
gcattagacccaaatgggacc

SEQ ID NO: 36 - DNA, 21 bp, synthetic construct
  source 1..21: mol_type = other DNA
gtgaaggctgtttccaaagag

SEQ ID NO: 37 - DNA, 20 bp, synthetic construct
  source 1..20: mol_type = other DNA
ggaggcatgctctccaagcc

SEQ ID NO: 38 - DNA, 20 bp, synthetic construct
  source 1..20: mol_type = other DNA
gctcctcatttggctcttcg

SEQ ID NO: 39 - DNA, 21 bp, synthetic construct
  source 1..21: mol_type = other DNA
gctcatttggctcttcgaggt

SEQ ID NO: 40 - DNA, 26 bp, synthetic construct
  source 1..26: mol_type = other DNA
agggcctacctcgaagagccaaatga

SEQ ID NO: 41 - DNA, 31 bp, synthetic construct
  source 1..31: mol_type = other DNA
ggcccagggcctacctcgaagagccaaatga

SEQ ID NO: 42 - DNA, 30 bp, synthetic construct
  source 1..30: mol_type = other DNA
agggcctacctcgaagagccaaatgaggag

SEQ ID NO: 43 - DNA, 26 bp, synthetic construct
  source 1..26: mol_type = other DNA
gcccagggcctacctcgaagagccaa

SEQ ID NO: 44 - DNA, 31 bp, synthetic construct
  source 1..31: mol_type = other DNA
agctggcccagggcctacctcgaagagccaa

SEQ ID NO: 45 - DNA, 30 bp, synthetic construct
  source 1..30: mol_type = other DNA
gcccagggcctacctcgaagagccaaatga

SEQ ID NO: 46 - DNA, 20 bp, synthetic construct
  source 1..20: mol_type = other DNA
gctgctgtagtagctcagca

SEQ ID NO: 47 - DNA, 21 bp, synthetic construct
  source 1..21: mol_type = other DNA
gccctgcagccacgcacccac

SEQ ID NO: 48 - DNA, 21 bp, synthetic construct
  source 1..21: mol_type = other DNA
gcgacacccatggagttcatg

SEQ ID NO: 49 - DNA, 21 bp, synthetic construct
  source 1..21: mol_type = other DNA
gccttaccctctacccccagc

SEQ ID NO: 50 - DNA, 20 bp, synthetic construct
  source 1..20: mol_type = other DNA
gaggagcgtgacgacaccca

SEQ ID NO: 51 - DNA, 21 bp, synthetic construct
  source 1..21: mol_type = other DNA
gttagacccaaatgggaccgg

SEQ ID NO: 52 - DNA, 21 bp, synthetic construct
  source 1..21: mol_type = other DNA
gacaggttgagctcctccgtc

SEQ ID NO: 53 - DNA, 20 bp, synthetic construct
  source 1..20: mol_type = other DNA
ggaggcatgctctccaagcc

SEQ ID NO: 54 - DNA, 21 bp, synthetic construct
  source 1..21: mol_type = other DNA
gcgcccccattagacccaaat

SEQ ID NO: 55 - DNA, 21 bp, synthetic construct
  source 1..21: mol_type = other DNA
gctacctcgaagagccaaatg

SEQ ID NO: 56 - DNA, 21 bp, synthetic construct
  source 1..21: mol_type = other DNA
gctctacccccagctggccca

SEQ ID NO: 57 - DNA, 20 bp, synthetic construct
  source 1..20: mol_type = other DNA
ggctgctgtagtagctcagc

SEQ ID NO: 58 - DNA, 21 bp, synthetic construct
  source 1..21: mol_type = other DNA
gctctacccccagctggccca

SEQ ID NO: 59 - DNA, 35 bp, synthetic construct
  source 1..35: mol_type = other DNA
gctcttcgaggtaggccctgggccagctgggggta

SEQ ID NO: 60 - DNA, 30 bp, synthetic construct
  source 1..30: mol_type = other DNA
tcgaggtaggccctgggccagctgggggta

SEQ ID NO: 61 - DNA, 20 bp, synthetic construct
  source 1..20: mol_type = other DNA
gacggaggagctcaacctgt

SEQ ID NO: 62 - DNA, 20 bp, synthetic construct
  source 1..20: mol_type = other DNA
gagctcaacctgtgggtgcg

SEQ ID NO: 63 - DNA, 21 bp, synthetic construct
  source 1..21: mol_type = other DNA
gcctgtgggtgcgtggctgca

SEQ ID NO: 64 - DNA, 21 bp, synthetic construct
  source 1..21: mol_type = other DNA
gcagcagcctcatgaactcca

SEQ ID NO: 65 - DNA, 20 bp, synthetic construct
  source 1..20: mol_type = other DNA
gtgtcgtcacgctcctcatt

SEQ ID NO: 66 - DNA, 21 bp, synthetic construct
  source 1..21: mol_type = other DNA
gctgggccagctgggggtaga

SEQ ID NO: 67 - DNA, 21 bp, synthetic construct
  source 1..21: mol_type = other DNA
gccagctgggggtagagggta

SEQ ID NO: 68 - DNA, 21 bp, synthetic construct
  source 1..21: mol_type = other DNA
gtagagggtaaggagagcctc

SEQ ID NO: 69 - DNA, 20 bp, synthetic construct
  source 1..20: mol_type = other DNA
gagagcctccggtcccattt

SEQ ID NO: 70 - DNA, 21 bp, synthetic construct
  source 1..21: mol_type = other DNA
gcggtcccatttgggtctaat

SEQ ID NO: 71 - DNA, 21 bp, synthetic construct
  source 1..21: mol_type = other DNA
gccatttgggtctaatggggg

SEQ ID NO: 72 - DNA, 20 bp, synthetic construct
  source 1..20: mol_type = other DNA
ggtctaatgggggcggagcc

SEQ ID NO: 73 - DNA, 21 bp, synthetic construct
  source 1..21: mol_type = other DNA
gaatgggggcggagccaggct

SEQ ID NO: 74 - DNA, 21 bp, synthetic construct
  source 1..21: mol_type = other DNA
gacgctcctcatttggctctt

SEQ ID NO: 75 - DNA, 32 bp, synthetic construct
  source 1..32: mol_type = other DNA
gcccagggcctacctcgaagagccaaatgagg

SEQ ID NO: 76 - DNA, 27 bp, synthetic construct
  source 1..27: mol_type = other DNA
tggcctacctcgaagagccaaatgagg

SEQ ID NO: 77 - DNA, 23 bp, synthetic construct
  source 1..23: mol_type = other DNA
gcctacctcgaagagccaaatga

SEQ ID NO: 78 - DNA, 26 bp, synthetic construct
  source 1..26: mol_type = other DNA
agggcctacctcgaagagccaaatga

SEQ ID NO: 79 - DNA, 30 bp, synthetic construct; identical to SEQ ID NO: 45
  source 1..30: mol_type = other DNA

SEQ ID NO: 80 - DNA, 32 bp, synthetic construct
  source 1..32: mol_type = other DNA
tggcccagggcctacctcgaagagccaaatga

SEQ ID NO: 81 - DNA, 35 bp, synthetic construct
  source 1..35: mol_type = other DNA
agctggcccagggcctacctcgaagagccaaatga

SEQ ID NO: 82 - DNA, 24 bp, synthetic construct
  source 1..24: mol_type = other DNA
gcctacctcgaagagccaaatgag

SEQ ID NO: 83 - DNA, 27 bp, synthetic construct
  source 1..27: mol_type = other DNA
agggcctacctcgaagagccaaatgag

SEQ ID NO: 84 - DNA, 31 bp, synthetic construct
  source 1..31: mol_type = other DNA
gcccagggcctacctcgaagagccaaatgag

SEQ ID NO: 85 - DNA, 33 bp, synthetic construct
  source 1..33: mol_type = other DNA
tggcccagggcctacctcgaagagccaaatgag

SEQ ID NO: 86 - DNA, 36 bp, synthetic construct
  source 1..36: mol_type = other DNA
agctggcccagggcctacctcgaagagccaaatgag

SEQ ID NO: 87 - DNA, 25 bp, synthetic construct
  source 1..25: mol_type = other DNA
gcctacctcgaagagccaaatgagg

SEQ ID NO: 88 - DNA, 28 bp, synthetic construct
  source 1..28: mol_type = other DNA
agggcctacctcgaagagccaaatgagg

SEQ ID NO: 89 - DNA, 32 bp, synthetic construct; identical to SEQ ID NO: 75
  source 1..32: mol_type = other DNA

SEQ ID NO: 90 - DNA, 34 bp, synthetic construct
  source 1..34: mol_type = other DNA
tggcccagggcctacctcgaagagccaaatgagg

SEQ ID NO: 91 - DNA, 37 bp, synthetic construct
  source 1..37: mol_type = other DNA
agctggcccagggcctacctcgaagagccaaatgagg

SEQ ID NO: 92 - DNA, 26 bp, synthetic construct
  source 1..26: mol_type = other DNA
gcctacctcgaagagccaaatgagga

SEQ ID NO: 93 - DNA, 29 bp, synthetic construct
  source 1..29: mol_type = other DNA
agggcctacctcgaagagccaaatgagga

SEQ ID NO: 94 - DNA, 33 bp, synthetic construct
  source 1..33: mol_type = other DNA
gcccagggcctacctcgaagagccaaatgagga

SEQ ID NO: 95 - DNA, 35 bp, synthetic construct
  source 1..35: mol_type = other DNA
tggcccagggcctacctcgaagagccaaatgagga

SEQ ID NO: 96 - DNA, 38 bp, synthetic construct
  source 1..38: mol_type = other DNA
agctggcccagggcctacctcgaagagccaaatgagga

SEQ ID NO: 97 - DNA, 27 bp, synthetic construct
  source 1..27: mol_type = other DNA
gcctacctcgaagagccaaatgaggag

SEQ ID NO: 98 - DNA, 30 bp, synthetic construct; identical to SEQ ID NO: 42
  source 1..30: mol_type = other DNA

SEQ ID NO: 99 - DNA, 34 bp, synthetic construct
  source 1..34: mol_type = other DNA
gcccagggcctacctcgaagagccaaatgaggag

SEQ ID NO: 100 - DNA, 36 bp, synthetic construct
  source 1..36: mol_type = other DNA
tggcccagggcctacctcgaagagccaaatgaggag

SEQ ID NO: 101 - DNA, 39 bp, synthetic construct
  source 1..39: mol_type = other DNA
agctggcccagggcctacctcgaagagccaaatgaggag

SEQ ID NO: 102 - DNA, 28 bp, synthetic construct
  source 1..28: mol_type = other DNA
gcctacctcgaagagccaaatgaggagc

SEQ ID NO: 103 - DNA, 31 bp, synthetic construct
  source 1..31: mol_type = other DNA
agggcctacctcgaagagccaaatgaggagc

SEQ ID NO: 104 - DNA, 35 bp, synthetic construct
  source 1..35: mol_type = other DNA
gcccagggcctacctcgaagagccaaatgaggagc

SEQ ID NO: 105 - DNA, 37 bp, synthetic construct
  source 1..37: mol_type = other DNA
tggcccagggcctacctcgaagagccaaatgaggagc

SEQ ID NO: 106 - DNA, 40 bp, synthetic construct
  source 1..40: mol_type = other DNA
agctggcccagggcctacctcgaagagccaaatgaggagc

SEQ ID NO: 107 - DNA, 29 bp, synthetic construct
  source 1..29: mol_type = other DNA
gcctacctcgaagagccaaatgaggagcg

SEQ ID NO: 108 - DNA, 32 bp, synthetic construct
  source 1..32: mol_type = other DNA
agggcctacctcgaagagccaaatgaggagcg

SEQ ID NO: 109 - DNA, 36 bp, synthetic construct
  source 1..36: mol_type = other DNA
gcccagggcctacctcgaagagccaaatgaggagcg

SEQ ID NO: 110 - DNA, 38 bp, synthetic construct
  source 1..38: mol_type = other DNA
tggcccagggcctacctcgaagagccaaatgaggagcg

SEQ ID NO: 111 - DNA, 41 bp, synthetic construct
  source 1..41: mol_type = other DNA
agctggcccagggcctacctcgaagagccaaatgaggagcg

SEQ ID NO: 112 - DNA, 31 bp, synthetic construct
  source 1..31: mol_type = other DNA
gcctacctcgaagagccaaatgaggagcgtg

SEQ ID NO: 113 - DNA, 34 bp, synthetic construct
  source 1..34: mol_type = other DNA
agggcctacctcgaagagccaaatgaggagcgtg

SEQ ID NO: 114 - DNA, 38 bp, synthetic construct
  source 1..38: mol_type = other DNA
gcccagggcctacctcgaagagccaaatgaggagcgtg

SEQ ID NO: 115 - DNA, 40 bp, synthetic construct
  source 1..40: mol_type = other DNA
tggcccagggcctacctcgaagagccaaatgaggagcgtg

SEQ ID NO: 116 - DNA, 43 bp, synthetic construct
  source 1..43: mol_type = other DNA
agctggcccagggcctacctcgaagagccaaatgaggagcgtg

SEQ ID NO: 117 - DNA, 20 bp, synthetic construct
  source 1..20: mol_type = other DNA
gcagccacgcacccacaggt

SEQ ID NO: 118 - DNA, 21 bp, synthetic construct
  source 1..21: mol_type = other DNA
gaggagcgtgacgacacccat

SEQ ID NO: 119 - DNA, 20 bp, synthetic construct
  source 1..20: mol_type = other DNA
gacgacacccatggagttca

SEQ ID NO: 120 - DNA, 20 bp, synthetic construct
  source 1..20: mol_type = other DNA
gaagagccaaatgaggagcg

SEQ ID NO: 121 - DNA, 21 bp, synthetic construct
  source 1..21: mol_type = other DNA
gcattagacccaaatgggacc

SEQ ID NO: 122 - DNA, 21 bp, synthetic construct
  source 1..21: mol_type = other DNA
gtgaaggctgtttccaaagag

SEQ ID NO: 123 - DNA, 21 bp, synthetic construct
  source 1..21: mol_type = other DNA
gagcctggctccgcccccatt

SEQ ID NO: 124 - DNA, 20 bp, synthetic construct
  source 1..20: mol_type = other DNA
gcccccattagacccaaatg

SEQ ID NO: 125 - DNA, 20 bp, synthetic construct
  source 1..20: mol_type = other DNA
gagccaaatgaggagcgtga

SEQ ID NO: 126 - DNA, 105 bp, Homo sapiens
  source 1..105: mol_type = genomic DNA
gctgccctgctgagctactacagcagcctcatgaactccatgggtgtcgtcacgctcctcatttggctcttcgaggtaggccctgggccagctgggggtagaggg

SEQ ID NO: 127 - protein, 25 aa, Homo sapiens
AALLSYYSSLMDSMGVVTLLIWLFE

SEQ ID NO: 128 - DNA, 20 bp, synthetic construct
  source 1..20: mol_type = other DNA
gctcctcatttggctcttcg

SEQ ID NO: 129 - DNA, 20 bp, synthetic construct
  source 1..20: mol_type = other DNA
acgactcgatgatgtcgtcg

SEQ ID NO: 130 - DNA, 34 bp, synthetic construct
  source 1..34: mol_type = other DNA
gtgcgaggagtaaaccgagaagctctatccggga

SEQ ID NO: 131 - DNA, 40 bp, Homo sapiens
  source 1..40: mol_type = genomic DNA
cacgctcctcatttggctcttcgaggtaggccctgggcca

SEQ ID NO: 132 - DNA, 40 bp, Homo sapiens
  source 1..40: mol_type = genomic DNA
cacgctcctcatttggctcttcgagataggccctgggcca

SEQ ID NO: 133 - DNA, 40 bp, Homo sapiens
  source 1..40: mol_type = genomic DNA
cacgctcctcatttggctcttcgagataggccctggagct

SEQ ID NO: 134 - DNA, 40 bp, Homo sapiens
  source 1..40: mol_type = genomic DNA
cacgctcctcatttggctcttcgagataggccctggaccg

SEQ ID NO: 135 - DNA, 41 bp, Homo sapiens
  source 1..41: mol_type = genomic DNA
cacgctcctcatttggctcttcgagagctactacagcgagc

SEQ ID NO: 136 - DNA, 40 bp, Homo sapiens
  source 1..40: mol_type = genomic DNA
cacgctcctcatttggctcttcgagataggctactacagc

SEQ ID NO: 137 - DNA, 40 bp, Homo sapiens
  source 1..40: mol_type = genomic DNA
cacgctcctcatttggctcttcgagataggccctggctac

SEQ ID NO: 138 - DNA, 69 bp, Homo sapiens
  source 1..69: mol_type = genomic DNA
gggtgtcgtcacgctcctcatttggctcttcgaggcaggccctgggccagctgggggtagagggtaagg

SEQ ID NO: 139 - protein, 11 aa, Homo sapiens
GVVTLLIWLFE

SEQ ID NO: 140 - DNA, 51 bp, Homo sapiens
  source 1..51: mol_type = genomic DNA
tgggtgtcgtcacgctcctcatttggctcttcgaggtaggccctgggccag

SEQ ID NO: 141 - protein, 13 aa, Homo sapiens
MGVVTLLIWLFEV

SEQ ID NO: 142 - DNA, 43 bp, Homo sapiens
  source 1..43: mol_type = genomic DNA
tggctcttcgaggtaggccctgggccagctgggggtagagggt

SEQ ID NO: 143 - protein, 5 aa, Homo sapiens
WLFEV

SEQ ID NO: 144 - DNA, 20 bp, Homo sapiens
  source 1..20: mol_type = genomic DNA
caacctgtgggtgcgtggct

SEQ ID NO: 145 - DNA, 20 bp, Homo sapiens
  source 1..20: mol_type = genomic DNA
gctcttcgaggtaggccctg

SEQ ID NO: 146 - DNA, 20 bp, Homo sapiens
  source 1..20: mol_type = genomic DNA
ggggtagagggtaaggagag

SEQ ID NO: 147 - DNA, 20 bp, Homo sapiens
  source 1..20: mol_type = genomic DNA
gctcttcgaggttggccctg

SEQ ID NO: 148 - DNA, 28 bp, Homo sapiens
  source 1..28: mol_type = genomic DNA
ctcatttggctcttcgaggtaggccctg

SEQ ID NO: 149 - protein, 6 aa, Homo sapiens
LIWLFE

SEQ ID NO: 150 - DNA, 28 bp, Homo sapiens
  source 1..28: mol_type = genomic DNA
ctcatttggctcttcgagataggccctg

SEQ ID NO: 151 - DNA, 28 bp, Homo sapiens
  source 1..28: mol_type = genomic DNA
ctcatttggctcttcgaggcaggccctg

SEQ ID NO: 152 - DNA, 28 bp, Homo sapiens
  source 1..28: mol_type = genomic DNA
ctcatttggctcttcgaggttggccctg

SEQ ID NO: 153 - DNA, 61 bp, Homo sapiens
  source 1..61: mol_type = genomic DNA
tcttcgaggtaggccctgggccagctgggggtagaggtgaccattacaattggctgcgcta

SEQ ID NO: 154 - protein, 13 aa, Homo sapiens
LFEVGPGPAGGRG

SEQ ID NO: 155 - DNA, 34 bp, Homo sapiens
  source 1..34: mol_type = genomic DNA
tcttcgaggtgaccattacaattgggctgcgcta

SEQ ID NO: 156 - DNA, 63 bp, Homo sapiens
  source 1..63: mol_type = genomic DNA
tcttcgagataggccctgggccagctgggggtagagggtgaccattacaattgggctgcgcta

SEQ ID NO: 157 - DNA, 63 bp, Homo sapiens
  source 1..63: mol_type = genomic DNA
tcttcgaggttggccctgggccagctgggggtagagggtgaccattacaattgggctgcgcta

SEQ ID NO: 158 - protein, 7 aa, Homo sapiens
PLQLGCA

SEQ ID NO: 159 - DNA, 44 bp, Homo sapiens
  source 1..44: mol_type = genomic DNA
tggctcttcgagataggccctgggccagctgggggtagaggtga

SEQ ID NO: 160 - protein, 14 aa, Homo sapiens
WLFEIGPGPAGGRG

SEQ ID NO: 161 - DNA, 44 bp, Homo sapiens
  source 1..44: mol_type = genomic DNA
tggctcttcgagttaggccctgggccagctgggggtagaggtga

SEQ ID NO: 162 - protein, 14 aa, Homo sapiens
WLFELGPGPAGGRG

SEQ ID NO: 163 - DNA, 45 bp, Homo sapiens
  source 1..45: mol_type = genomic DNA
tggctcttcgaggcaggccctgggccagctgggggtagagggtga

SEQ ID NO: 164 - protein, 14 aa, Homo sapiens
WLFEAGPGPAGGRG

SEQ ID NO: 165 - DNA, 44 bp, Homo sapiens
  source 1..44: mol_type = genomic DNA
tggctcttcgaggttggccctgggccagctgggggtagaggtga

SEQ ID NO: 166 - protein, 14 aa, Homo sapiens
WLFEVGPGPAGGRG

SEQ ID NO: 167 - DNA, 43 bp, Homo sapiens
  source 1..43: mol_type = genomic DNA
atttggctcttcgaggtaggccctgggccagctgggggtagag

SEQ ID NO: 168 - protein, 5 aa, Homo sapiens
IWLFE

SEQ ID NO: 169 - DNA, 43 bp, Homo sapiens
  source 1..43: mol_type = genomic DNA
atttggctcttcgagataggccctgggccagctgggggtagag

SEQ ID NO: 170 - DNA, 59 bp, Homo sapiens
  source 1..59: mol_type = genomic DNA
tttggctcttcgagataggccctgggccagctgggggtagagggtgaccattacaattg

SEQ ID NO: 171 - protein, 15 aa, Homo sapiens
IWLFEIGPGPAGGRG

SEQ ID NO: 172 - protein, 4 aa, Homo sapiens
PLQL

SEQ ID NO: 173 - DNA, 59 bp, Homo sapiens
  source 1..59: mol_type = genomic DNA
aattgntnttngagataggccctgggccagctggcggnagagggtgaccattacaattg

SEQ ID NO: 174 - DNA, 58 bp, Homo sapiens
  source 1..58: mol_type = genomic DNA
taagtctatgngagataggccnngggccagttgggtgtaggggtgaccattacaattg

SEQ ID NO: 175 - DNA, 30 bp, Homo sapiens
  source 1..30: mol_type = genomic DNA
tttggctctttgaggtgaccattacaattg